(19) United States
(12) Patent Application Publication
(10) Pub. No.: US 2003/0130566 A1
(43) Pub. Date: Jul. 10, 2003

(54) Methods and systems for using visual imagery and utilitarian articles to promote or demonstrate emotional and/or physical responses

(76) Inventor: Gary J. Hawkes, London (GB)

Correspondence Address: GREENBERG TRAURIG, P.C., Suite 2500, 77 West Wacker Drive, Chicago, IL, 60601, US

(21) Appl. No.: 10/302,532

(22) Filed: Nov. 21, 2002

(30) Foreign Application Priority Data
Dec. 4, 2001 (GB) 0129024.6

(51) Int. Cl. A61B 5/00

(52) U.S. Cl. 600/300

(57) ABSTRACT

A method for influencing mental or physical states of an individual, using visual imagery, wherein the visual imagery is created using source image components derived from physical structures or materials that are causally associated with, the mental or physical state that is sought to be influenced, or which is desired to be created. The present invention is also directed to the visual images that are created, as well as to articles bearing the visual images that have been created, for purposes of exercising the method.

### BACKGROUND OF THE INVENTION

1. Field of the Invention

The present invention is directed to methods and apparatus for presenting visual images, in the form of poster-like displays, or decoration on utilitarian or ornamental objects, or the like, wherein the visual images are composed in whole or in part, of image components created from illustrations of chemicals, biological or otherwise, or of other materials, that are representative of or in some other logical or causal way connected to or associated with a desired effect to be produced in, on or by a user.

2. Prior Art

Methods and apparatus involving the use of visual images, for the purpose of altering a person's physical, emotional or mental state, are known. References disclosing such methods and apparatus include:

Greenwood, U.S. Pat. No. 2,283,628 discloses a sleep aid device, incorporating the image of a sheep jumping a fence, mounted on a spinner.

Southwick, U.S. Pat. No. 3,340,865 discloses an apparatus for projecting differently colored lights, for purposes of permitting the detection of a person's “aural” colors, potentially for affecting a change in “feeling”.

Schoffer, U.S. Pat. No. 3,470,870 discloses an apparatus for inducing sleep or relaxation, employing light passing through a rotating multicolored disc.

Derouineau, U.S. Pat. No. 3,722,501 discloses a tranquilizing device, employing an internally illuminated rotating cylinder having openings therein for permitting escape of the light.

Dehlinger, U.S. Pat. No. 3,972,319 discloses glasses, having a movable mosaic pattern template mounted thereon. The template is configured to be moved, by the wearer's exhalations, thus creating a changing image pattern in the glasses.

Farley et al., U.S. Pat. No. 4,216,594 discloses a psychotherapeutic game employing image cards.

Filley, U.S. Pat. No. 4,388,918 discloses a mental harmonization process, involving the exposure of an individual to specifically selected colors and sounds.

Krane, U.S. Pat. No. 4,682,956 discloses a board game for determining personal traits and relationships between individuals in which characteristics are in part determined through the players' selection of color cubes.

Krane, U.S. Pat. No. 4,815,976 discloses a board game for determining personal traits and relationships between individuals in which characteristics are in part determined through the players' selection of color cubes.

Decker, U.S. Pat. No. 5,285,430 discloses a behavior modification wristwatch, in which positive reinforcement messages are programmed to be exhibited on a digital display.

Baccei et al., U.S. Pat. No. 5,371,627 discloses a method for making random dot stereograms.

Wexelman, U.S. Pat. No. 5,388,994 discloses a visual stimulation device incorporating a rotating disk, bearing colored regions, a mirror, pictures, text, etc. for treatment of psychological disorders.

Bielinski et al., U.S. Pat. No. 5,543,965 discloses a method and apparatus for producing random dot stereograms.

Schutz, U.S. Pat. No. 5,594,841 discloses methods for making stereograms.

Frenkel et al., U.S. Pat. No. 6,007,569 discloses methods and apparatus for determining the results of light therapies.

Sabuda, U.S. Pat. No. 6,048,301 discloses a method for stimulating biological processes, incorporating radiating a crystal, to generate a photon field, and exposing a subject within the photon field.

Koyama et al., U.S. Pat. No. 6,053,936 discloses an illumination device for modifying circadian rhythms, including a headband worn light source, and a control apparatus.

LaDue, U.S. Pat. No. 6,097,927 discloses a method for behavior modification using a computer, and employing virtual reality constructs, and further including biometric monitoring and recording.

Zaltman et al., U.S. Pat. No. 6,099,319 discloses a method and system for using neuroimaging as a marketing tool, specifically for using biometric data for validating whether a stimulus such as an advertisement evokes a particular mental response, such as emotion, preference or retention in memory.

Yu, U.S. Pat. No. 6,127,616 discloses a method for representing musical compositions using colors.

Pinter, U.S. Pat. No. 6,132,452 discloses a method for promoting energy flow in an organism, including mineral crystals installed in a cross-shaped structure.

Ark et al., U.S. Pat. No. 6,190,314 B1 discloses an input device with biometric sensors for sensing user emotions.

August, U.S. Pat. No. 6,254,427 B1 discloses an apparatus for promoting relaxation, etc., including the placement of an image, e.g., a landscape, onto a fabric for promoting an improved mental state.

Rose, U.S. Pat. No. 6,258,022 B1 is directed to a behavior modification method and apparatus, wherein a wearable apparatus is used to produce a perceptible stimulation which, during a prior hypnosis session, is correlated to improved performance, such as improved sporting performance.

LaDue, U.S. Pat. No. 6,259,889 B1 (division of U.S. Pat. No. 6,097,927) discloses a method for behavior modification using a computer, and employing virtual reality constructs, and further including biometric monitoring and recording.

Pinter, U.S. Pat. No. 6,277,142 B1 (continuation-in-part of U.S. Pat. No. 6,132,452) discloses a method for promoting energy flow in an organism, including dielectric materials and a plurality of conductive elements.

Olson et al., U.S. Pat. No. 6,297,424 B1 discloses a training diaper, with wetness indicating graphics, to provide motivational input to the wearer.

Blazey et al., U.S. Pat. No. 6,309,342 B1 discloses a method for determining the physiological reactivity of an individual with respect to images, including the measuring of biometric characteristics during a stress period and during a rest period, and measuring a biometric characteristic during an image presentation time.

Zaltman, U.S. Pat. No. 6,315,569 B1 discloses a method for conducting marketing research, incorporating biometric monitoring.

Weber et al., U.S. Pat. No. 6,352,528 B1 discloses a training diaper, with wetness indicating graphics, to provide motivational input to the wearer.

Freer, U.S. Pat. No. 6,402,520 B1 discloses a biofeedback system for improving learning skills.

### SUMMARY OF THE INVENTION

The present invention is directed in part to posters or other articles, bearing a unitary image or a composite image (which could be in any shape) made up of discrete image components. Each image component is, preferably, an illustration of a chemical compound (e.g., vitamin C, an amino acid, DNA), a cell structure, etc., that may present in the human body, when that person is experiencing a specifically identified emotional/mental/physical state, e.g., “love”. Alternatively, the chemical or other material, may be a substance that is known or suspected to be a causative agent, for prompting a change to a desired state, or for maintaining a desired state, or for preventing an undesired state.

The present invention is also directed in part, to a method for using the so-created visual imagery to prompt, promote, maintain, or prevent one or more identified emotional/mental/physical states. The nature (identity or provenance) of the composite image(s) would, preferably, be presented to the purchaser/consumer/user/observer of the poster or other article. This individual, being so made aware of the nature of the composite image and its constituent image components, would be prompted to attain, maintain, or avoid the specified mental/emotional/physical state, the “target” state being related, in a specified manner, to the nature of the constituent image components. As such, the poster or other article would be acting as a self-help aid device. According to some belief systems, the provenance of the image components, while useful, is not necessarily required. In this alternative interpretation of the “mode of operation” of this invention it is believed that there are inherent qualities (“vibrations”) relating to the nature and identity of the image components that would “resonate” in the individual observing/using/wearing/etc. the article upon which the image is placed, and thus is somehow affected by the image in some desired fashion.

One aspect of this invention, is the use of such images to specifically address attributes of physicality, namely sports performance. Garments, gear or the like, bearing composite images, again formed from image components illustrating various chemicals, etc., representative of or otherwise related to aspects of sports performance may be provided. For example, if the desired quality is speed or strength, then one such image component may be a chemical/crystalline photograph of a steroid, adrenaline, etc. Again, the desired result would be an enhancement of a particular sports performance characteristic (increased speed, strength, durability, or the like), through selection of the appropriate composite image and image components. The images may also be placed on (or more precisely, in) articles, such as in the linings of garments, so that while not publicly broadcast, the images are proximate to the wearer, and if known to the wearer, can have the inspirational “placebo” effect. Whether known to the wearer or not, the “resonance” effect, as described elsewhere herein may come into play.

In still another aspect of the invention, it is known that certain mental states can be correlated to colors (in that people will have different emotional responses to different colors/patterns, etc., and/or can be influenced to some degree, in different ways by different colors). For example, a suite of specifically color/pattern coordinated garments that may be used by an individual to “signal” to himself or to others either an emotional or mental state that he or she is in (e.g., a warning or other message) or to attempt to alter an existing mental state to a different desired state, through the wearing of garments/ornamentation/etc. in the color/pattern corresponding to the desired “target” state.

The invention comprises, in part, a method for influencing an emotional or physical state of an individual, using a visual image, comprising the steps of:

identifying a particular target state, being at least one of mental, emotional, physical, to be influenced;

identifying the desired manner in which the particular target state is to be influenced;

identifying at least one influence material which is one of causative of, resultant of, the target state;

making at least one visual representation of the at least one influence material;

creating at least one image component from each at least one visual representation of the at least one influence material;

creating a composite image from the at least one image component;

placing the composite image on an article;

bringing the individual to be influenced into contact with the article.

The step of identifying the desired manner in which the particular target state is to be influenced preferably further comprises the step of:

determining whether the influence sought is to be to maintain the target state, promote the target state, or prevent the target state.

The influence material may be one of:

a chemical which when brought into interactive contact with an individual directly influences the target state; a chemical which is found in an individual when that individual is experiencing the target state and which is characteristic of that target state; a cellular structure; an organism; an object.

The step of making at least one visual representation of the at least one influence material preferably further comprises the step of:

making a photographic representation of the at least one influence material.

The step of creating at least one image component from each at least one visual representation of the at least one influence material may further comprise the step of:

selectively altering the color values of the photographic representation.

The step of creating at least one image component from each at least one visual representation of the at least one influence material may further comprise the step of:

imposing a selected outline shape for the at least one image component.

The step of creating at least one image component from each at least one visual representation of the at least one influence material may further comprise the step of:

creating at least two image components from at least two visual representations of at least one influence material.

The step of creating a composite image may further comprise the step of:

selectively positioning the at least two image components relative to one another.

The step of creating at least one image component from each at least one visual representation of the at least one influence material alternatively may further comprise the step of:

creating at least two image components from at least two visual representations of at least two influence materials.

The step of creating a composite image further may further comprise the step of:

selectively positioning the at least two image components relative to one another; and

imposing a selected shape to the contours of the selectively positioned at least two image components, the selected shape having an association with the target state to be influenced.

The article upon which the composite image is to be placed preferably comprises one of:

a sheet of opaque display media; a sheet of transparent media; a sheet of translucent media; printing media; a garment; a structure; a vehicle; packaging for a commercial product; a visual prosthetic aid device.

The method may further comprise the step of:

associating verbal information content with the visual image, identifying the nature of the at least one image component.

The invention also comprises, in part, a method of preparation of a visual image for influencing an emotional or physical state of an individual, using the visual image, comprising the steps of:

identifying a particular target state, being at least one of mental, emotional, physical, to be influenced;

identifying the desired manner in which the particular target state is to be influenced;

identifying at least one influence material which is one of causative of, resultant of, the target state;

making at least one visual representation of the at least one influence material;

creating at least one image component from each at least one visual representation of the at least one influence material;

creating a composite image from the at least one image component.

The invention also comprises, in part, an article bearing a visual image for influencing an emotional or physical state of an individual, using the visual image, comprising:

a physical object;

a composite image disposed thereon,

the composite image including at least one image component,

the at least one image component including at least one visual representation of an influence material,

the at least one influence material being one of causative of, resultant of, a target state of an individual which is to be influenced,

the target state of the individual which is to be influenced, being one of mental, emotional, physical.

The physical object is preferably one of:

a sheet of opaque display media; a sheet of transparent media; a sheet of translucent media; printing media; a garment; a structure; a vehicle; packaging for a commercial product; a visual prosthetic aid device.

The at least one influence material is preferably one of:

a chemical which when brought into interactive contact with an individual directly influences the target state; a chemical which is found in an individual when that individual is experiencing the target state and which is characteristic of that target state; a cellular structure; an organism; an object.

The methods may further comprise one of the following steps:

projecting the composite image onto an article;

publishing the composite image in a printed medium;

recording the composite image onto videotape;

broadcasting the composite image via one of television, cable, satellite communication;

communicating the composite image via email;

locating the composite image in an internet website.

The composite image may be one of: a representation of a human body part; a stylized design; an animal; a plant; a geographic structure; an abstract geometric figure.

### BRIEF DESCRIPTION OF THE DRAWINGS

FIG. 1 is a collective photograph of several image component for materials associated with the emotional state of “love”.

FIG. 2 is an enlarged view of an exemplary photograph of the image component estradiol.

FIG. 3 is an enlarged view of an exemplary photograph of the image component oxytocin.

FIG. 4 is an enlarged view of an exemplary photograph of the image component serotonin.

FIG. 5 is an enlarged view of an exemplary photograph of the image component noradrenaline.

FIG. 6 is an enlarged view of an exemplary photograph of the image component beta-endorphin.

FIG. 7 is an enlarged view of an exemplary photograph of the image component vasopressin.

FIG. 8 is an enlarged view of an exemplary photograph of the image component testosterone.

FIGS. 9 and 10 are further exemplary photographs of vasopressin.

FIGS. 11 and 12 are further exemplary photographs of oxytocin.

FIGS. 13 and 14 are further exemplary photographs of serotonin.

FIGS. 15 and 16 are further exemplary photographs of betaendorphin.

FIGS. 17 and 18 are further exemplary photographs of noradrenaline.

FIGS. 19 and 20 are further exemplary photographs of testosterone.

FIGS. 21 and 22 are exemplary photographs of dopamine, also a “love” related compound.

FIG. 23 is a collective photograph of various materials that are associated with the realm of “sport”.

FIG. 24 is an enlarged view of an exemplary photograph of the image component testosterone.

FIG. 25 is an enlarged view of an exemplary photograph of the image component Creatine.

FIG. 26 is an enlarged view of an exemplary photograph of the image component co-enzyme Q.

FIG. 27 is an enlarged view of an exemplary photograph of the image component Vitamin E.

FIG. 28 is an enlarged view of an exemplary photograph of the image component Vitamin C.

FIG. 29 is an enlarged view of an exemplary photograph of the image component ADENOSINE TRIPHOSPHATE.

FIG. 30 is an enlarged view of an exemplary photograph of the image component glucose.

FIG. 31 is a collective collage photograph of various materials associated with specific aspects of the realm of “sport”.

FIG. 32 is an exemplary photograph of the AIDS virus.

FIG. 33 is an exemplary photograph of asbestos.

FIG. 34 is an exemplary photograph of a cancerous cell.

FIG. 35 is a composite image in the form of an eye.

FIG. 36 is another composite image in the form of an eye.

FIGS. 37-45 represent various forms of possible representative composite images.

### DETAILED DESCRIPTION OF THE DRAWINGS

While this invention is susceptible of embodiment in many different forms, there is shown in the drawings and will be described herein in detail, a specific embodiment, with the understanding that the present invention is to be considered as an exemplification of the principles of the invention and is not intended to limit the invention to the embodiment illustrated.

The present invention is directed to the creation and deployment of images, which may be displayed on a variety of media, for the purposes of achieving an effect upon a person, to influence that person's physical, mental or emotional state.

There exists a well-known phenomenon, commonly called the “placebo effect”. The underlying principle of this phenomenon, is that persons (test subjects) are given some form of treatment (typically some form of medicine or food supplement), which, the test subjects are told, is being administered in order to achieve some desired (usually) beneficial effect. Some of the test subjects will be given an “actual” chemical substance, the efficacy of which is the subject of the test. Others of the test subjects, while being told they are being given the actual chemical substance, are actually being given what is essentially an inert or neutrally-acting substance, at least from the standpoint of the desired effect.

What has been noted is that sometimes the persons being given the “fake” treatment nevertheless experience the desired effect. Sometimes the number of incidences of persons experiencing a desired result, without the benefit of the treatment, is so great that mere statistical anomaly cannot adequately account for the result. Whether the person's belief in the efficacy of the drug had some sort of influence (so-called “mind over matter” type situations) is subject to much debate. Nevertheless, the phenomenon has been frequently observed in a variety of situations.

There is a widely held belief system that includes the concept that various substances or cellular structure, organism or objects, typically chemicals that are either representative of certain physical or mental states, or are used to alter physical or mental states, or to treat illnesses, etc., have some indefinable characteristic that may extend beyond the mere physicality of the substance or cellular structure, organism or object, and that this indefinable characteristic (“vibration” or “resonance”).

It is further conjectured that such “vibrations” or “resonance” even extends to abstract or graphic representations of such substances or cellular structure, organism or objects, such that exposure to such representations may achieve effects similar to that achieved by exposure to the substances or cellular structure, organism or objects themselves.

It is desired, in the present invention, to make advantageous use and application of these phenomena, to bring about improvement in quality of life; treatment of illness and management of health; improvement in performance, whether mental or physical; and improvement in mental, physical or emotional states.

The means to bringing this goal about is accomplished through the following series of steps:

1. Identify the desired resultant effect.

2. Identify the chemical substances, cellular structures, organisms or objects, etc., that either are causative agents for bringing about the desired resultant influence or effect, or are themselves “effects” or characteristic evidence of the desired resultant effect. For the purposes of this application, such chemicals, cellular structures, organisms or objects may be referred to collectively as “influence materials”.

3. Make photographic representations of one or more of the influence materials, that have been identified in step 2.

4. Using one or more of the representations from step 3 as image “components”, create a composite image (e.g., a shape may be superimposed on the basic image component, to provide a secondary or overarching image, like an eye or stylized heart).

5. Place the composite image on a medium configured to expose the desired “target” to the image, for delivery of the “content” of the image.

By discussing the method, step by step, different embodiments of the invention, and different fields of application can be described.

Identify the desired resultant effect. The resultant effect may be generally classified as either mental or physical, with “emotional” being a subset of “mental”. One may be seeking to achieve an influence or impact, such as a change, improvement or enhancement, in one's physical condition (general or specific physical health issues), physical performance under specific or general conditions (strength, speed, stamina, resistance to bruising, cramping, performance induced injury, etc.), or in one's emotional state, or general mental health or performance (memory, speed of recall). One may be seeking to improve running speed, have a better general outlook on life, or have improved concentration under stress, among a nearly infinite number of other potential desired results.

The desired influence or “effect” may be to transmit, express, promote, suppress, and/or reinforce an emotional or mental state. For example, one may wish to express emotional feelings like love, hate, anger, joy, bemusement, humor, etc. The goal may be to express to others the emotional or mental state, toward imparting that condition or influencing others, or for having an effect upon oneself.

Alternatively, one may be trying to cause a physical result, such as by the wearing of a garment or ornamentation, containing an image that is correlated with a physical characteristic or performance, such as an image of adrenaline (for strength and speed) or an image of a healing or health-related agent (e.g., a homeopathic material such as arnica, or a compound such as vitamin C).

The identified “state” may be either positive or negative. That is, a positive emotional state like “love” or a negative emotional state like “hate” or “anger”; a positive physical state (like a healthy cell) or a negative physical state (a diseased cell, e.g., cancer). The “negative” states may be used in an image in a non-negative way, such as in “avoidance of” that negative state.

Identify the causative or characteristic chemical substances, cellular structures, organisms or objects, etc. (the “influence materials”).

The various “states” that are mentioned above have associations with certain substances, cellular structures, organisms, etc. (collectively “influence materials”). These associations may be causal in nature (i.e., the influence material either causes or impacts upon the target “state”). Alternatively, the influence material may be resultant in nature, e.g., evidence of the target “state” (e.g., extreme emotion can cause the release of adrenaline in the bloodstream).

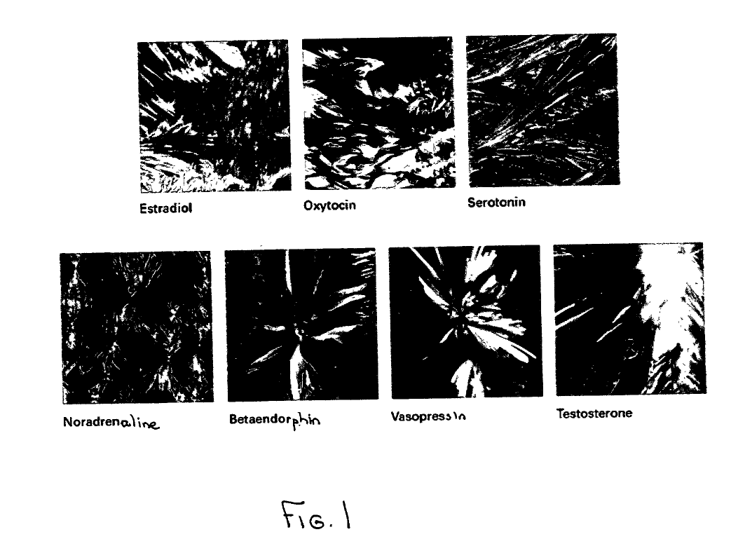

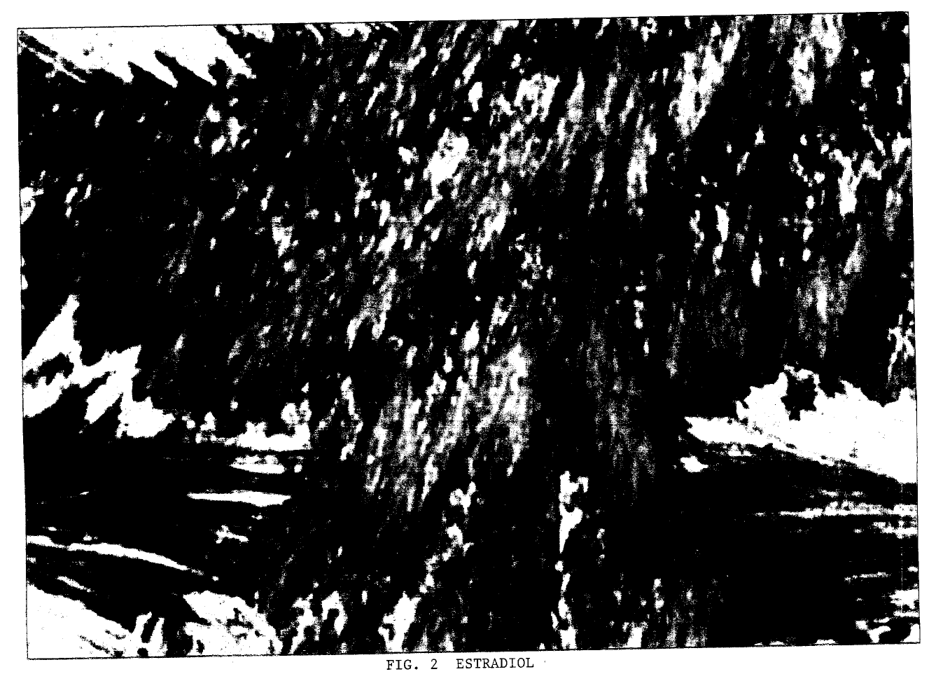

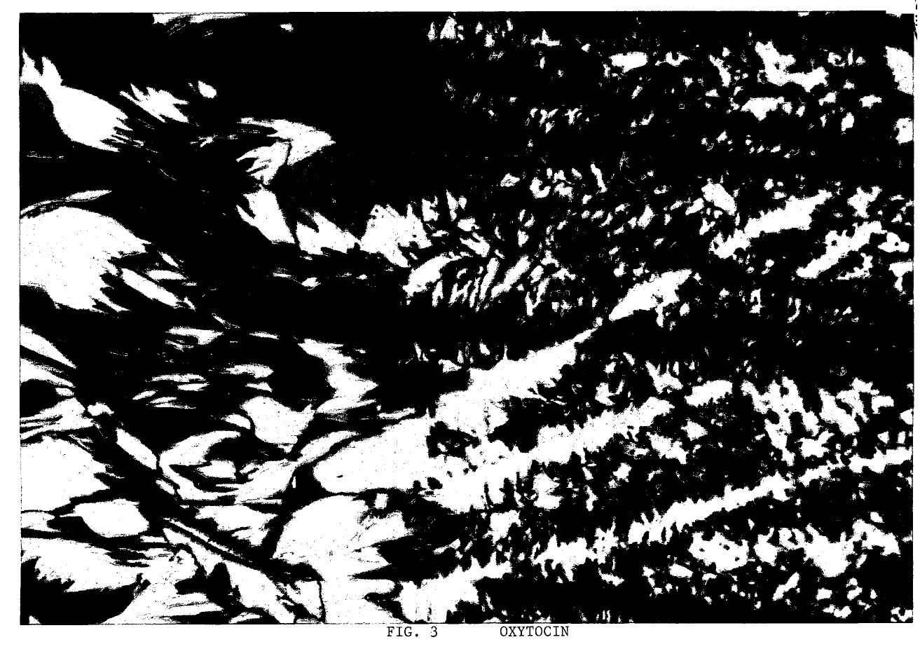

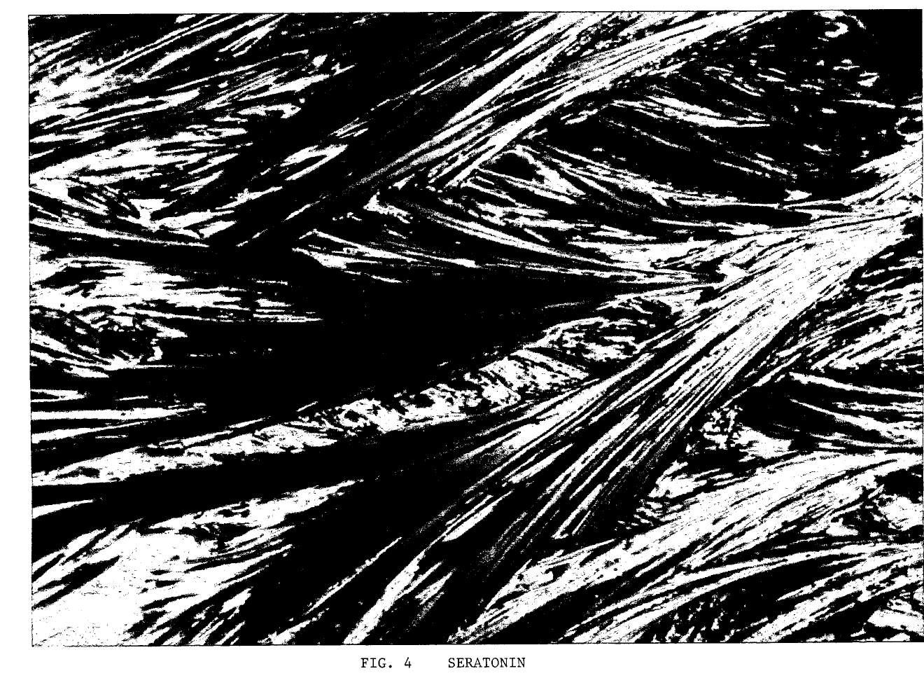

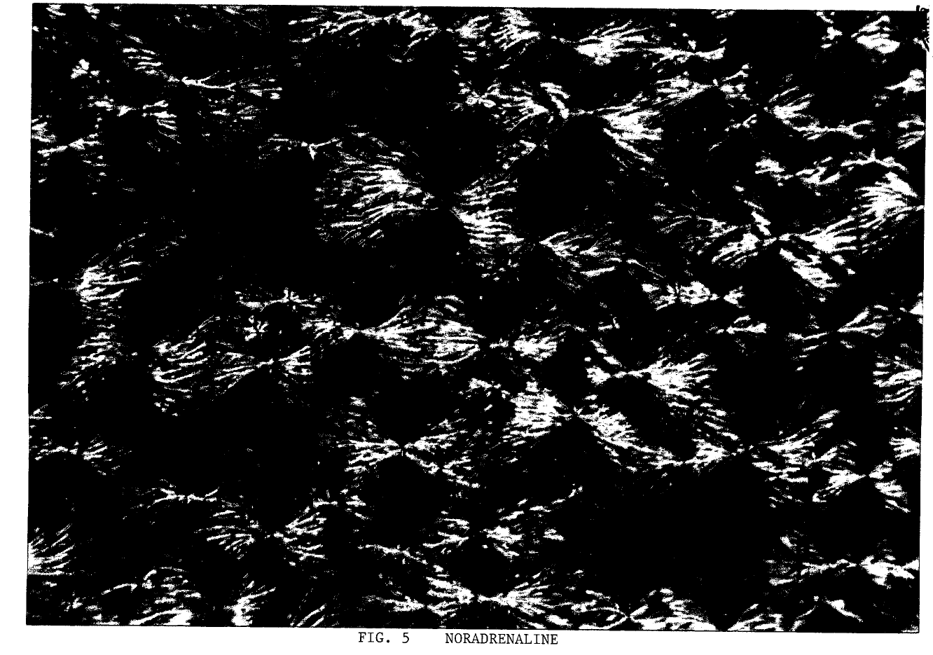

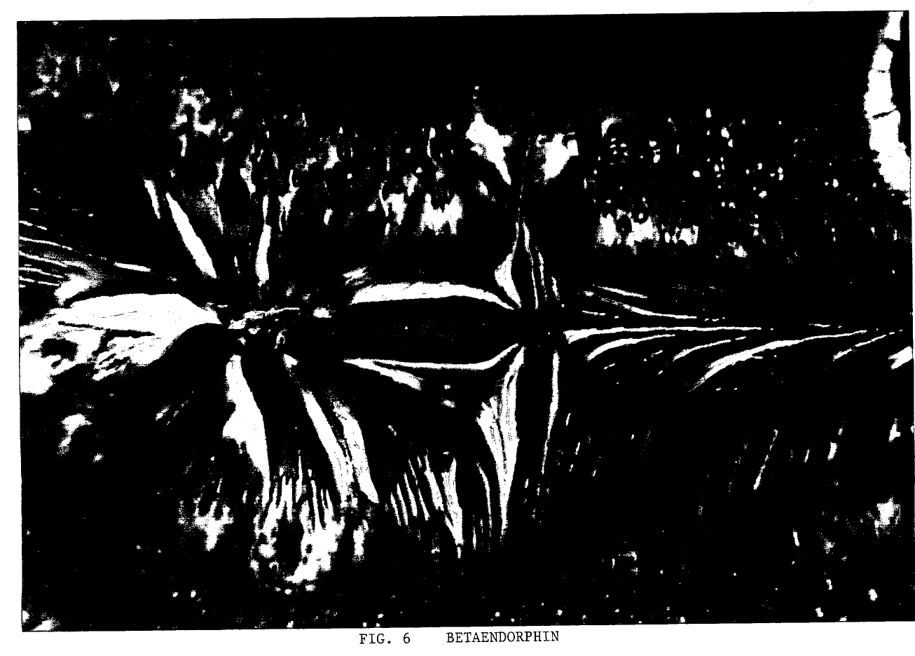

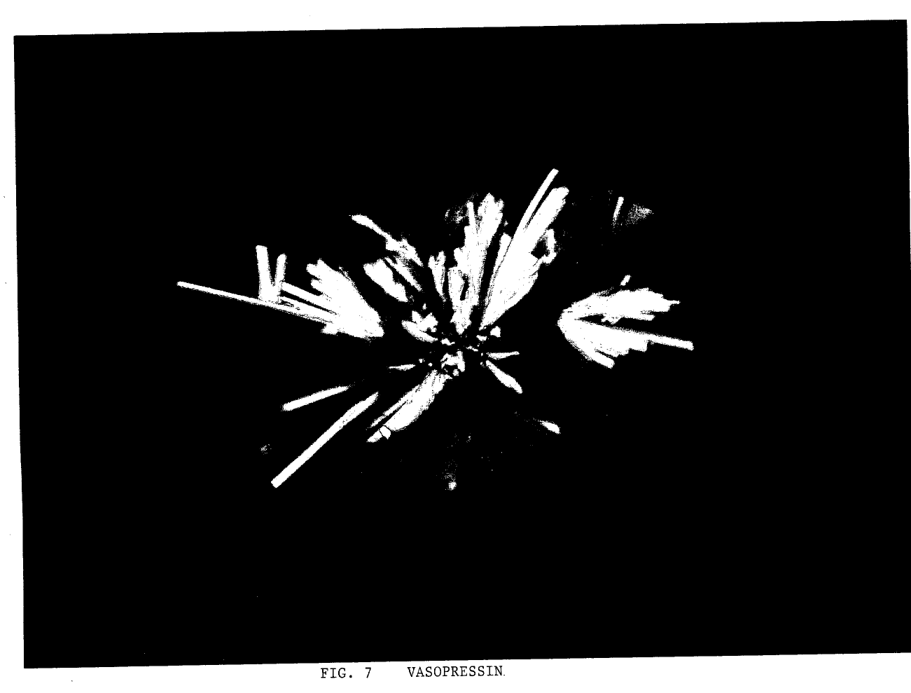

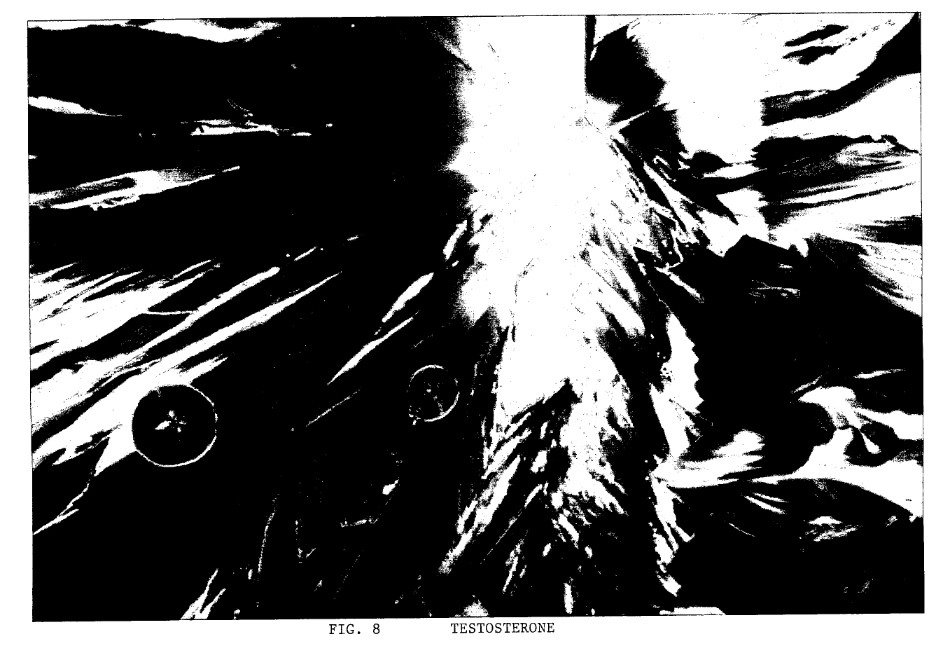

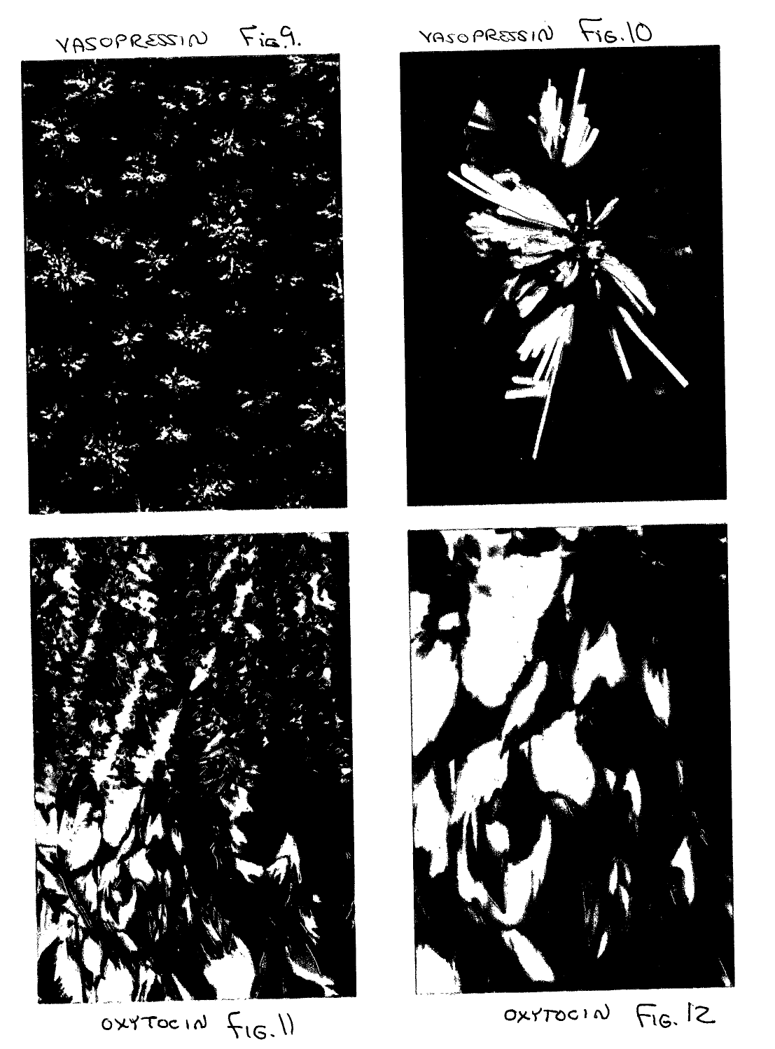

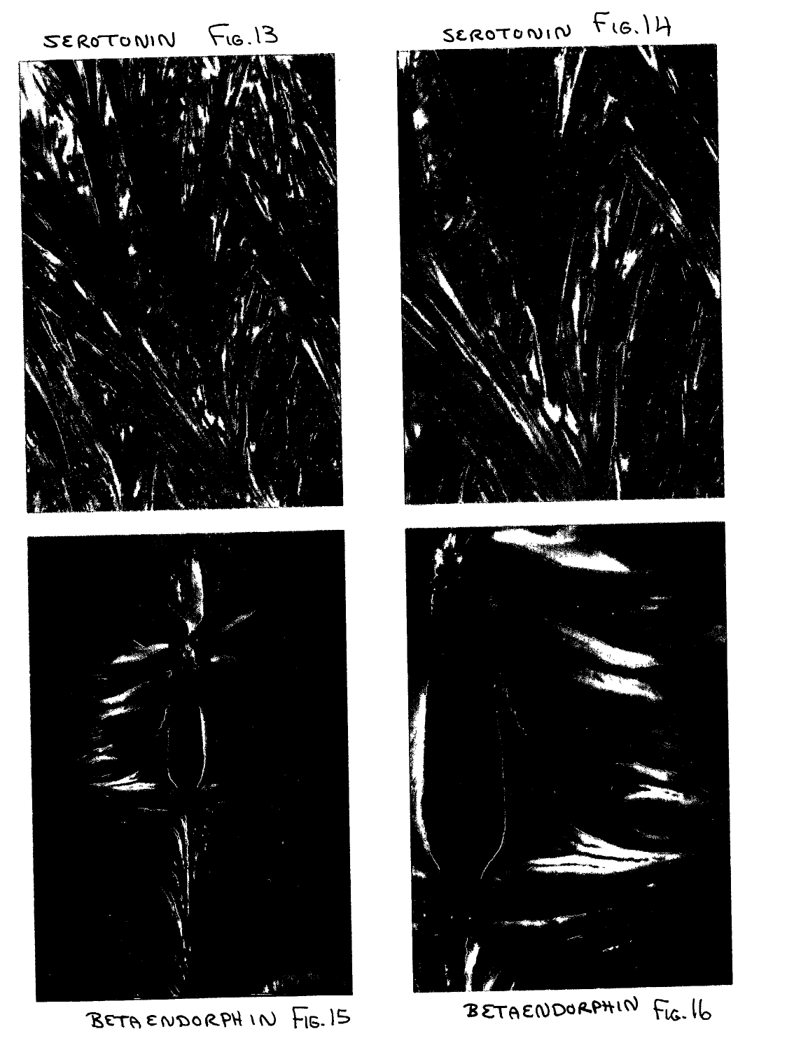

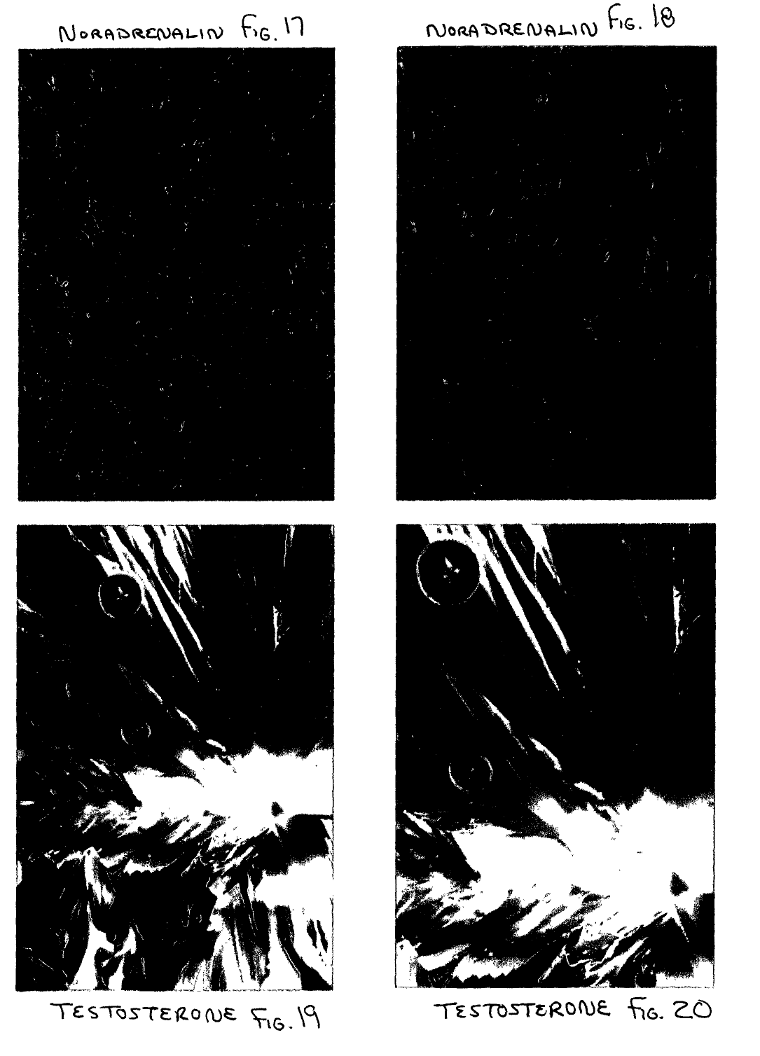

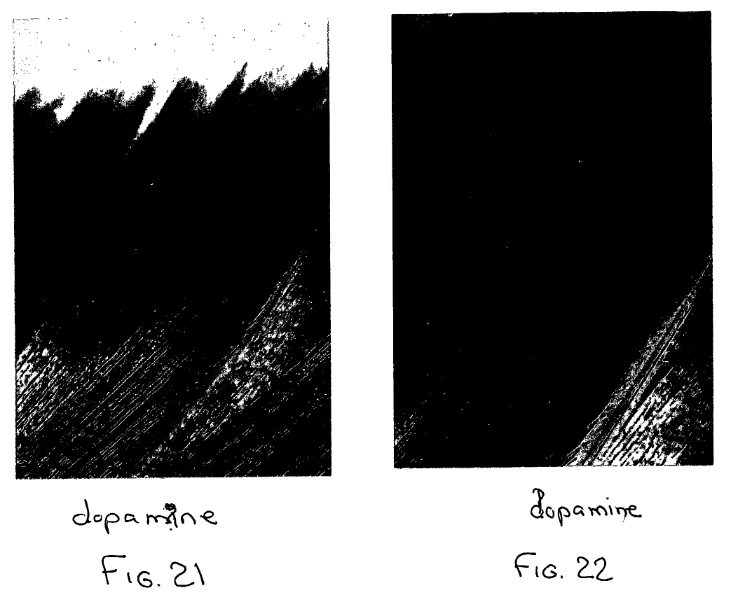

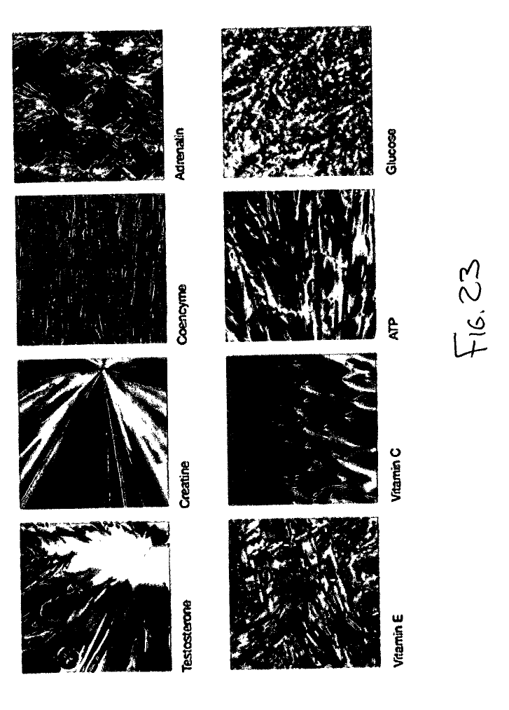

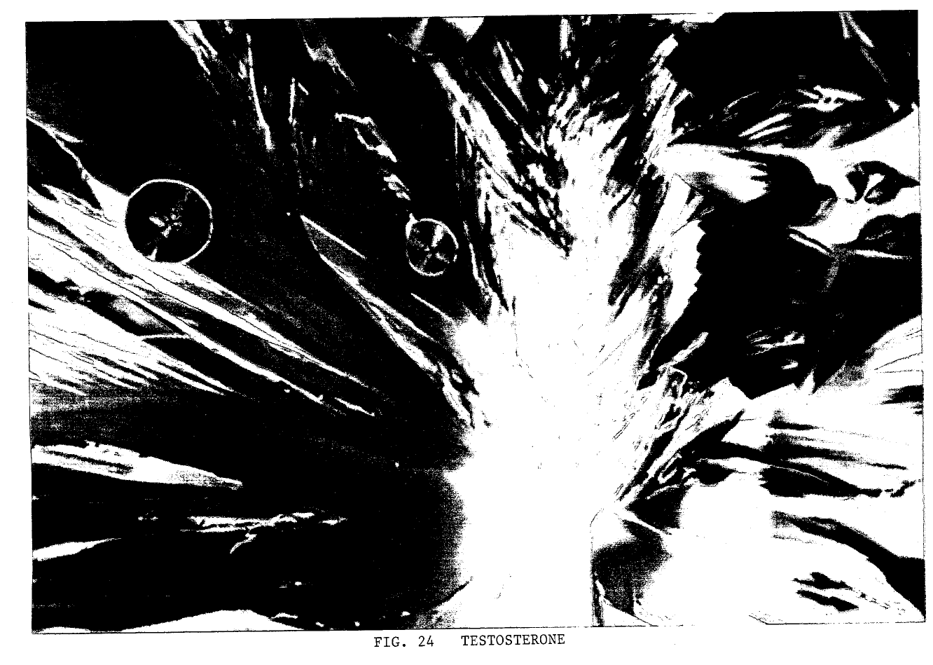

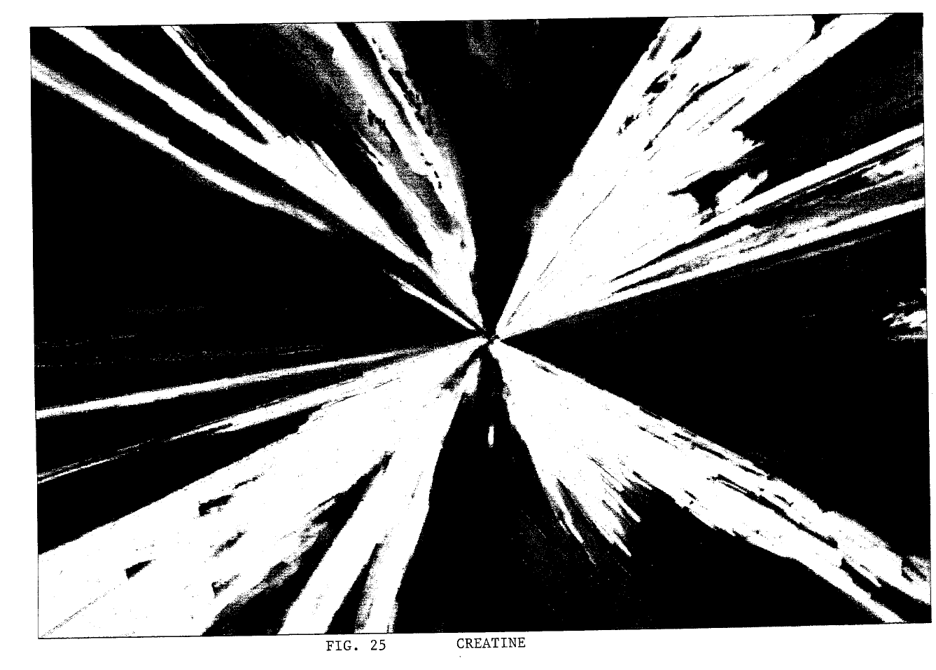

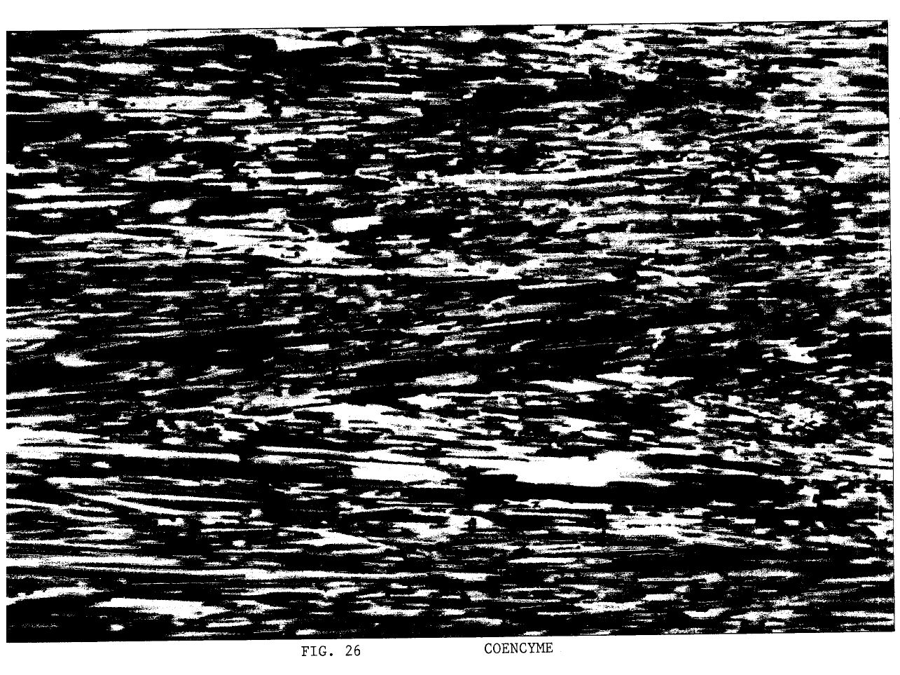

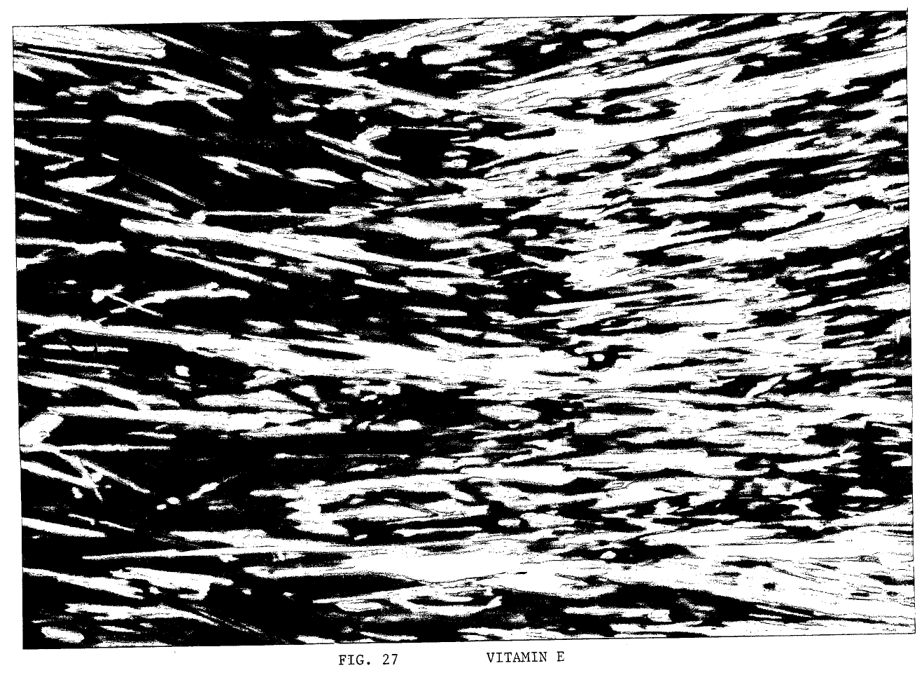

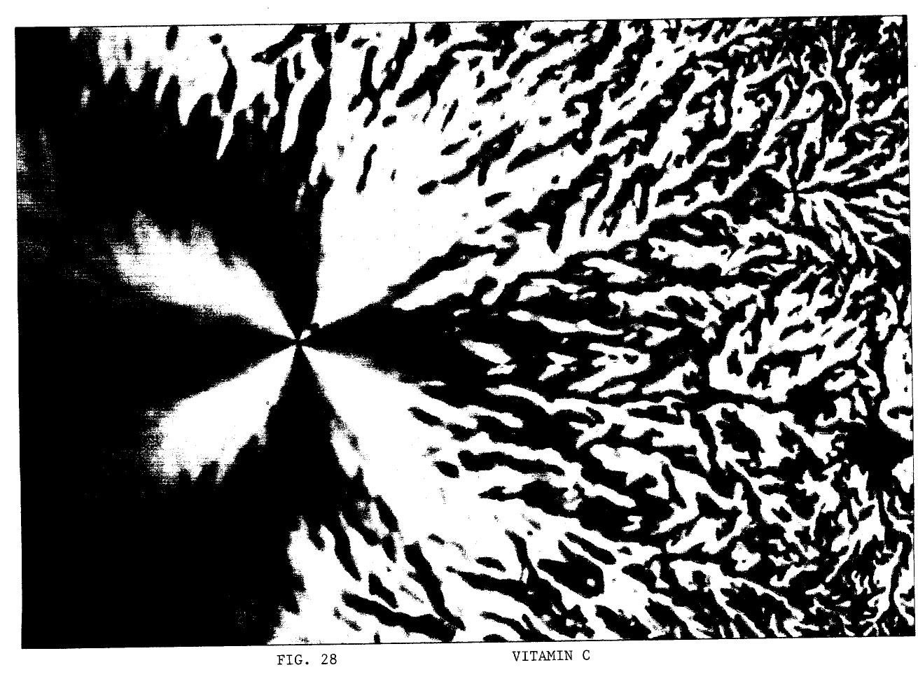

Taking for example, the emotional state of “love”, there have been a number of chemical compounds that have been determined to be present in a human organism, at the time that that organism has been, as well as can be determined, found to be experiencing feelings corresponding to those of one or more of the collection of emotional states referred to as “love”. Among these chemicals are: estradiol (“estrogen”); seratonin; noradrenaline; beta-endorphin; testosterone; vasopressin; oxytocin; dopamine. See FIGS. 1, 21 and 22. There are of course, many more. However, certain research has suggested that these chemicals have particular significance, and so have been selected as being the representative image components relevant to this “state”. Other materials may be selected, provided that they in fact have a relationship with the state of “love” (i.e., are found, singly or in combination, in the biochemistry of a person experiencing the “state”).

For example, oxytocin and vasopressin, FIGS. 3, 7, 11, 12, 9, 10, are found in the limbic system, often referred to as “the emotional brain”. These two substances play a part both in sexual and non-sexual love, although they may act differently in males and females, oxytocin believed to be more pertinent to “love” in females and vasopressin believed to be more pertinent to “love” in males. Oxytocin has been conjectured to induce nurturing-type love in males. Both chemical systems appear to establish attachment bonds between mother and child, and also to impact love, friendship and societal preferences among adults. Oxytocin is also believed to have anti-aggressive properties.

Noradrenaline and dopamine (FIGS. 5, 21, 22, 17, 18) are believed to have an impact in pair-bonding, which may be reinforced in interactions with oxytocin.

Endorphins (FIGS. 6, 15, 16) have mood elevating properties, are naturally produced pain killers and have been implicated in the creation of emotional attachment.

Seratonin (FIGS. 4, 13, 14) is a neurochemical system which modulates emotional states.

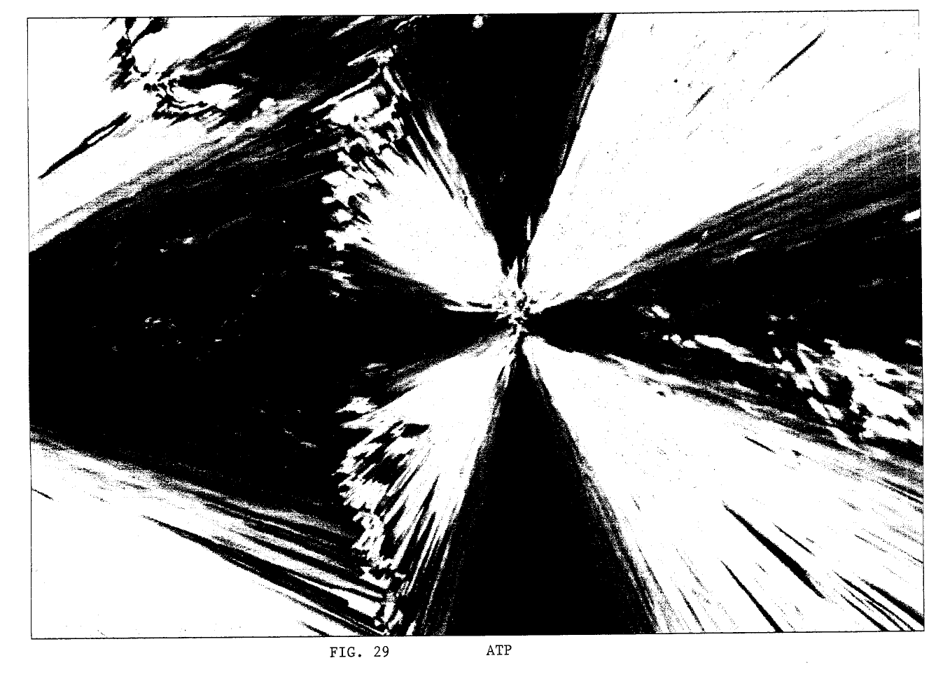

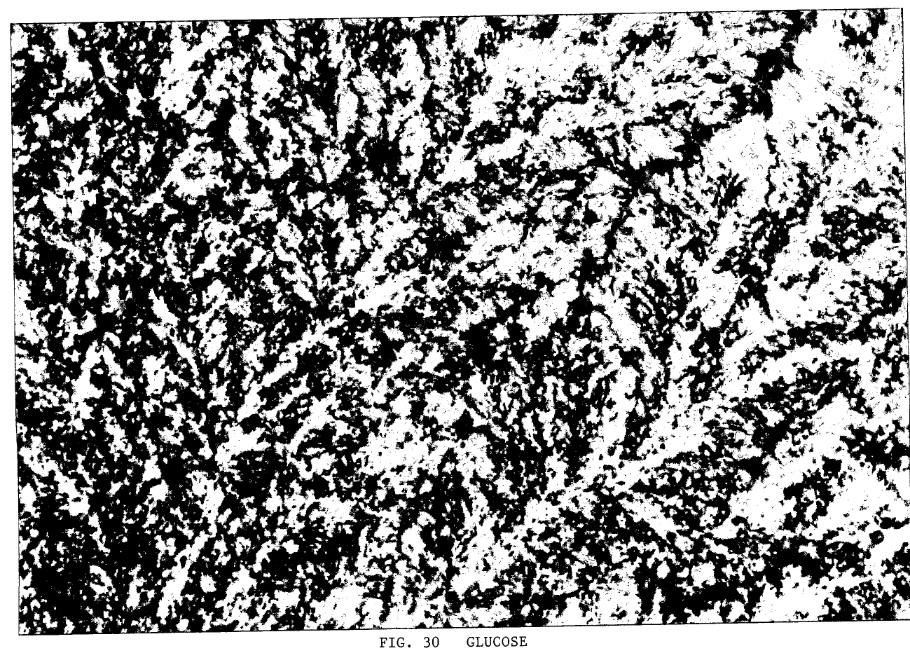

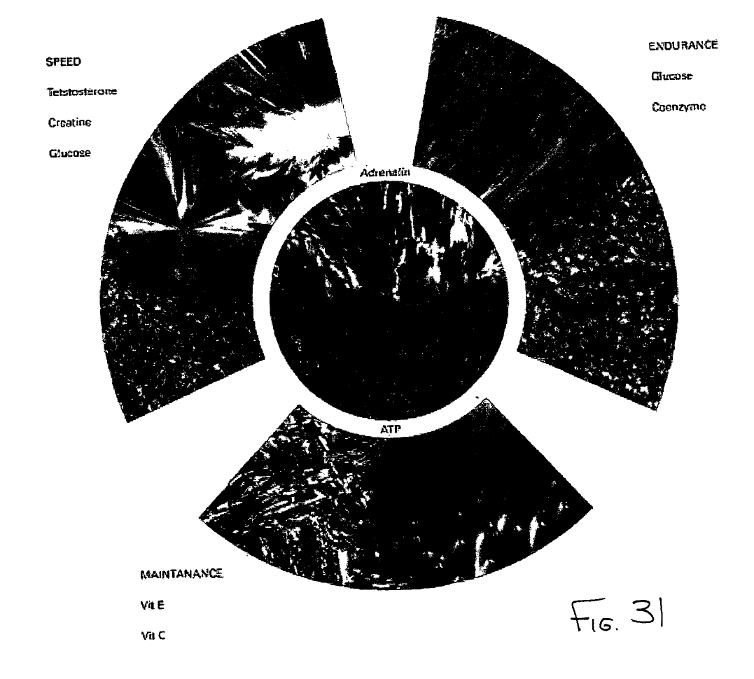

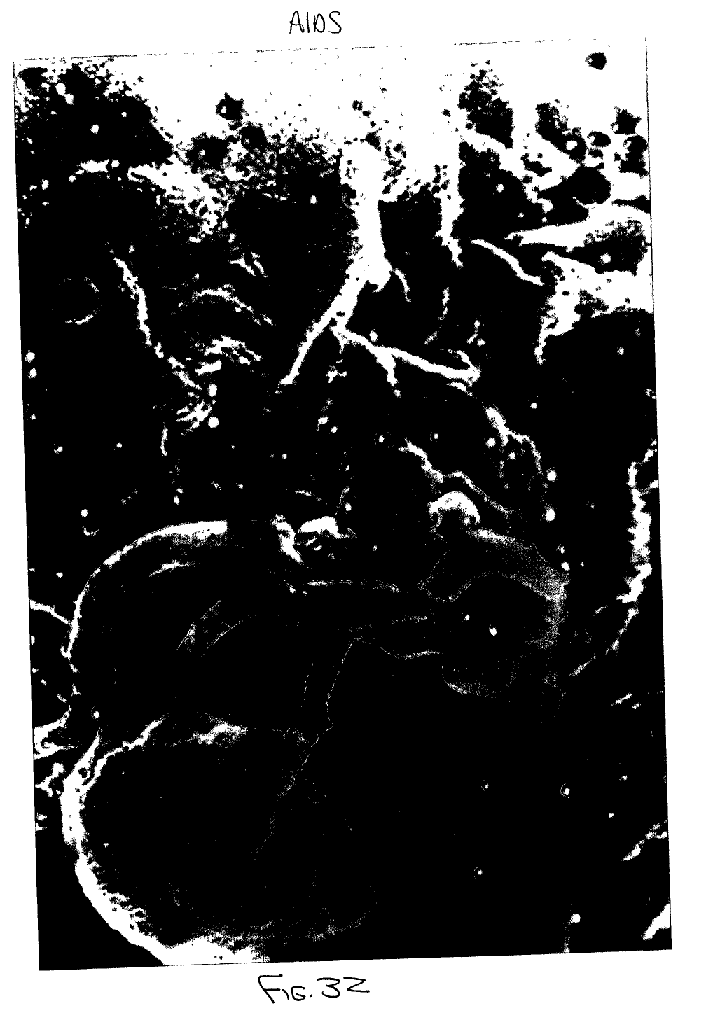

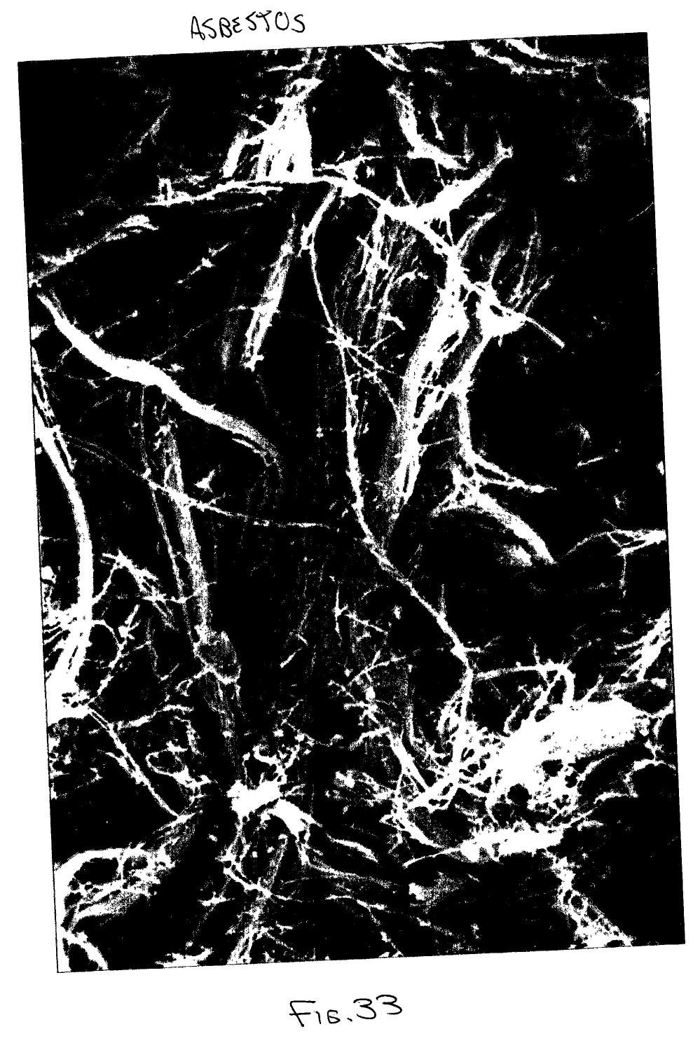

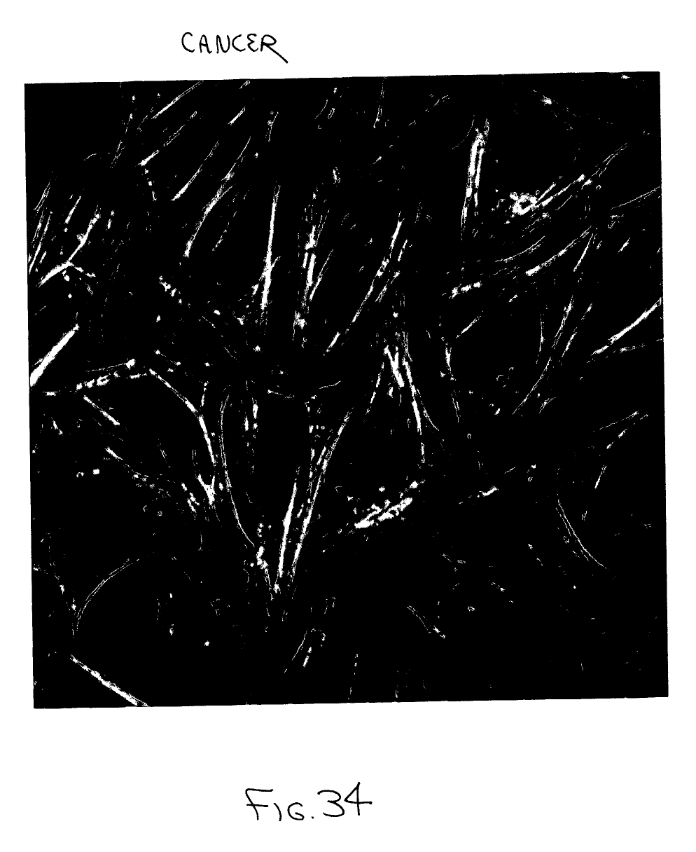

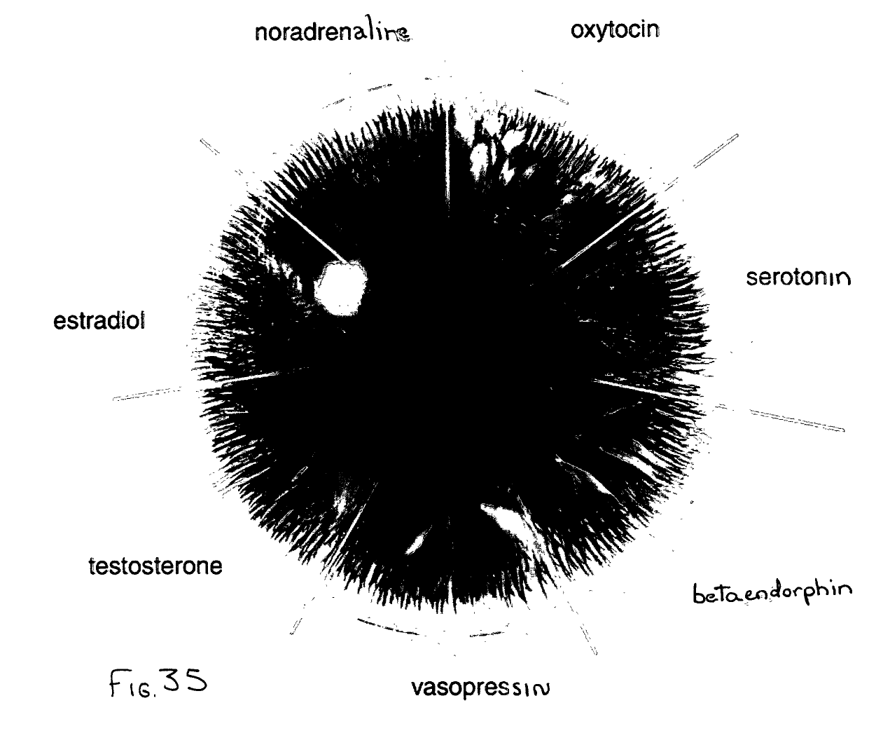

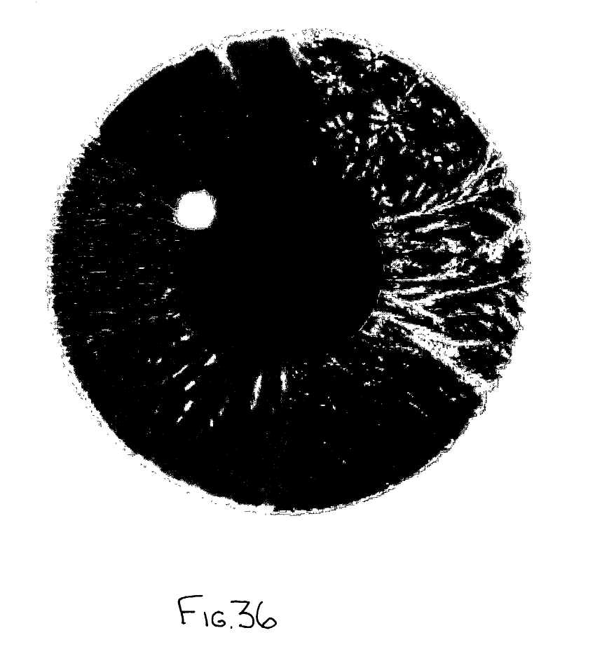

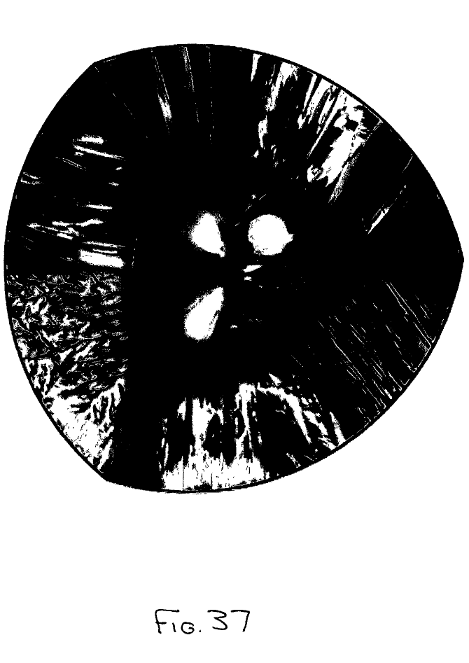

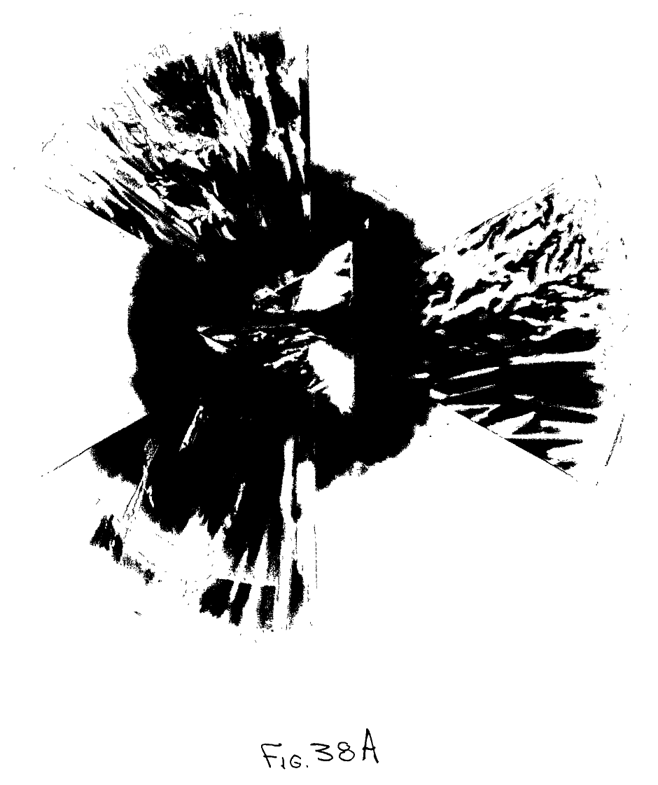

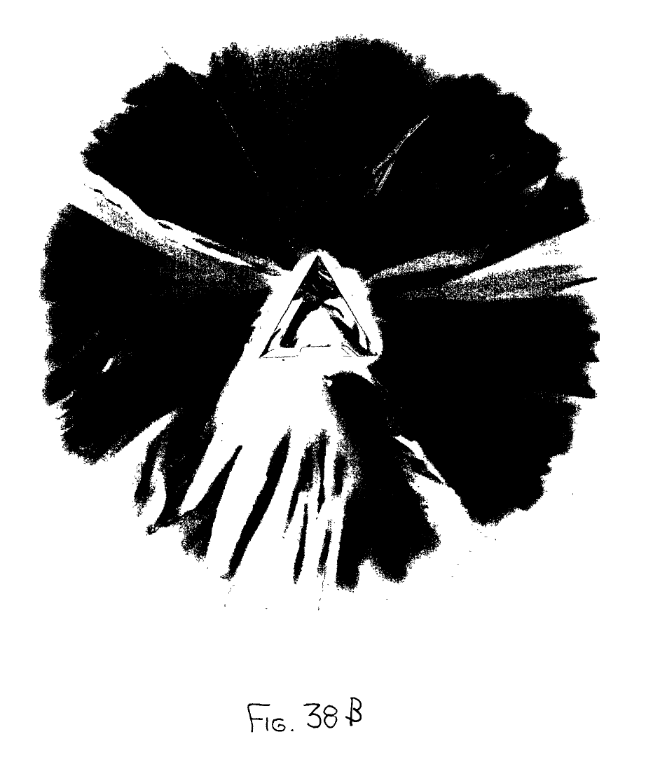

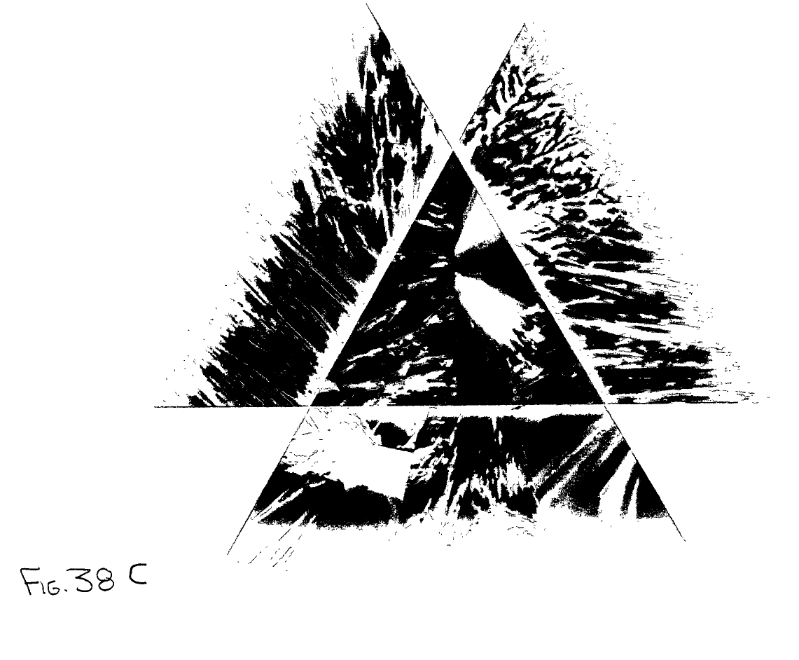

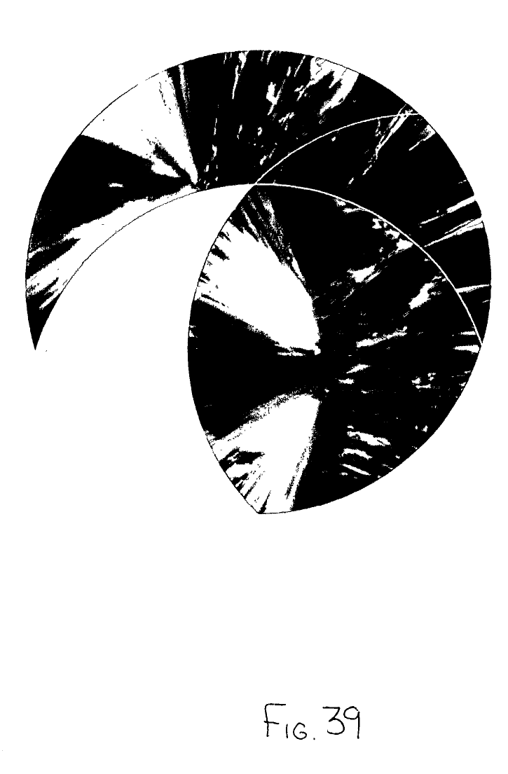

The realm of “sport” (i.e., physical endeavors of all sorts) is also a likely objective for application of the principles of the present invention. A sports enthusiast may be seeking to affect his performance in any of a number of ways: speed, endurance, maintenance of a particular level of conditioning, etc. See FIG. 31. Some of the many relevant compounds include testosterone, Creatine, co-enzyme Q (2,3-Dimethoxy-5-Methyl-1,4-Benzoquinone), adrenaline, Vitamin E, Vitamin C, ADENOSINE TRIPHOSPHATE, and glucose. See FIGS. 24-30.

The determination of “characteristic” chemicals, compounds, organisms, cellular structures, etc. may be accomplished through ordinary clinical testing techniques, and using normal laboratory protocols, so the specific details of such methods are readily understood by those of ordinary skill in the art of biochemistry, and psychology, and need not be expounded upon in detail here.

Other “states” and materials with which they are associated are:

Pleasure/Euphoria: dopamine, noradrenaline, beta-endorphins, endogenous cannabinoids (THC).

Stress: cortisol (increased level in blood, during stress), adrenocorticotropic hormone (ACTH—releases the cortisol); and corticotropin releasing hormone (CRH—releases ACTH—also associated with anxiety and depression).

Anxiety/Fear: GABA (anti-anxiety chemical—responsive to anxiety reducing drugs such as benzodiazepines (e.g., valium) and SSRI's (e.g., Prozac), and alcohol; serotonin.

Aggression: testosterone (causes it); serotonin (controls it).

Sleep (and jet lag): light (controls circadian rhythm; ameliorates effects of jet lag); vasopressin (regulates sleep); interleukin-1 (stimulates immune system); adenosine (increases sleep); caffeine.

Learning/memory: NMDA; glutamate.

Pain: bradykinin (sensitivity), prostaglandins (sensitivity), substance P (causes release of histamine—for pain perception); capsaicin (causes release of substance P in mouth); endorphins.

Hunger, appetite, control of eating and weightdieting: leptin (released by fat cells in bloodstream—causes nerve cells to release peptide neurotransmitters ALPHA-MSH and CART [cocaine and amphetamine-regulated transcript])—which diminish appetite); ACTH and thyroid stimulating hormone (TSH); neuropeptide Y and AgRP (agouti-related peptide)—stimulate food intake; cholecystokinin (appetite suppressant).

There are also chemicals, substances, or cellular structure, organism or objects, which are causative agents with respect to a particular desired result. For a very basic example, one can use common aspirin, which has a direct causal link to relief from a headache (even if no one really knows how or why headaches go away in the presence of aspirin).

The following is a chart indicating various physical and mental states, activities and the like, and various materials that are associated or are believed to be related to, causative of, caused by, etc., those states, activities, etc., in which the aforementioned materials and states are included. This chart is by no means exhaustive nor is it intended to be so, but is merely exemplary of types of conditions, and the materials believed to be correlated to those conditions.

Sport:

Speed

ATP (Adenosine Triphosphate)

Noradrenaline (and/or Adrenaline)

CP (Creatine Phosphate)

Testosterone

Glucose (or Glycogen)

Endurance

ATP (Adenosine Triphosphate)

Noradrenaline (and/or Adrenaline)

Co-enzyme Q (also called Ubiquinone)

Glucose (and/or Glycogen)

Recovery

ATP (Adenosine Triphosphate)

Noradrenaline (and/or Adrenaline)

Vitamin C

Vitamin E

Other Sport Chemicals

HGH (Human Growth Hormone) also called Somatotropin

Endorphins e.g. Beta Endorphin

Corticosteroids e.g. Cortisone

ADP (Adenosine Diphosphate)

AMP (Adenosine Monophosphate)

Pyruvate

Fatty Acids e.g. Triglycerides

Proteins e.g. Alanine

Hb (Haemoglobin)

Myoglobin

EPO (Erythroprotein)

2,3-DPG (2,3-Diphosphoglycerate)

Myosin

Actin

Actinase

Prolactin

Insulin

Glucagon

Cortisol (also called Hydrocortisone)

Vitamins especially B6

Phosphofructokinase

Lactic acid

Lactate

[Possible image Mitochondria, fast/slow twitch muscle fibers.]

Sport:

Banned Sports Substances: (as per the UK Sport Anti-Doping Directorate)

Peptide Hormones, Mimetics & analogues (& all releasing factors):

[Prohibited in males only]

GH (Growth Hormone)

Corticotrophin

Chorionic Gonadotrophin

Pituitary & synthetic Gonadotrophins

Next 2 only allowed to treat certified insulin dependent diabetes

EPO (Erythroprotein)

Insulin

Stimulants:

Amphetamine

Bromantan

Caffeine (above 12 mcg/ml)

Cocaine

Ephedrine (above 10 mcg/ml)

Methylphenidate (above 10 mcg/ml)

Pseudoephedrine (above 25 mcg/ml)

Phenylephrine

Phenylpropanolamine (above 25 mcg/ml)

Salbutamol (allowed by inhaler when conditions satisfied)

Salmeterol (allowed by inhaler when conditions satisfied)

Terbutaline (allowed by inhaler when conditions satisfied)

certain beta2agonists

Narcotics:

Morphine

Diamorphine (Heroin)

Pethidine

Methadone

Anabolic Agents:

Androstenedione

Clenbuterol

DHEA

Methandienone

Nandrolone

19 nor-steroids

Stanozolol

Testosterone

certain beta2agonists

Diuretics:

Bendrofluazide

Frusemide

Mannitol (prohibited by IV injection)

Triamterene

Hydrochlorothiazide

Sport:

Other Prohibited Substances in Certain Circumstances:

ALCOHOL/Cannabinoids

Local Anesthetics

Glucocorticosteroids

Beta-Blockers

Love:

Primary 7

Serotonin

Noradrenaline

Dopamine

Beta-Endorphin

Testosterone

Vasopressin

Oxytocin

Other Love Chemicals

Alpha MSH

Estrogen

Progesterone

LHRH (Lutenising Hormone Releasing Hormone)

FSH (Follicle Stimulating Hormone)

Diet:

Diet Suppressants

Gherelin

Leptin

Insulin

Beta Endorphin

Pro-Opiomelanocortin

Alpha-MSH

Serotonin

CART (Cocaine and Amphetamine-Regulated Transcript)

ACTH (AdrenoCortico Tropic Hormone)

TSH (Thyroid Stimulating Hormone)

CCK (Cholecystokinin)

Promoting Eating

Neuropeptide Y

AgRP (Agouti-Related Peptide)

Gherelin

Orexin

Sleep:

Sleep Chemicals

Serotonin

Vasopressin

GABA

CALB (Calbindin)

AVP (Arginine Vasopressin)

A II (Angiotensin)

mENK (Met-Enkephalin)

GAL (Galanin)

VIP (Vasoactive Intestinal Polypeptide)

GRP (Gastrin Releasing Peptide)

NT (Neurotensin)

NPY (Neuropeptide Y)

5HT (5-Hydroxytryptamine)

Melatonin

Jet Lag

GABA

Vasopressin

mENK (Met-Enkephalin)

Serotonin

Insomnia

Interleukin-1

Adenosine

Hair Loss:

Minoxidil

Sex:

Arousal

Vasopressin

Oxytocin

LHRH (Lutenising Hormone Releasing Hormone)

Endorphins

Orgasm

Vasopressin

Oxytocin

Endorphins

Fertility

Male sperm count

Female ova production

Relaxation:

GABA

benzodiazepines e.g. Valium, Mogadon

Stress:

CRF (Corticotropin Releasing Factor)

CRH (Corticotropin Releasing Hormone)

ACTH (AdrenoCortico Tropic Hormone)

Noradrenaline

Corticosterone

Cortisol

Learning, Memory, Concentration, Alertness:

Glutamate

ACTH (AdrenoCortico Tropic Hormone)

Vasopressin

Prolactin

TRH (Thyroid Releasing Hormone)

ACh (Acetycholine)

Nitric Oxide

Estrogen

Memory

NMDA

Glutamate

Alertness

Acetylcholine

Noradrenaline

Serotonin

Caffeine

Happiness/Pleasure:

Opiates e.g. Endorphin

Serotonin

Dopamine

Noradrenaline

Beta Endorphin

THC (active substance in Cannabis)

Anandamide

Anxiety:

Benzodiazepines

Valium

SSRI's (Selective Serotonin Reuptake Inhibitors)

Prozac

Alcohol

GABA

Serotonin

Barbiturates

Alcohol

Medical Applications:

Anesthetics/Pain Control

Endorphins

Dynorphin

Increasing Pain

Bradykinin

Prostaglandins

Substance P

Histamine

Blood Clotting

Fibrinogen

Healing Wounds

Substance P

Prostaglandins

Serotonin

Histamine

Glutamate

Oxytocin

Bone formation

Ageing

Reduction of HGH (Human Growth Hormone)

Reduction of HGHRH (Human Growth Hormone Releasing Hormone)

DHEA

Steroids:

Natural

Artificial

Banned Substances:

Drugs:

Marijuana

Cannabis

Speed

Cocaine

Heroin

Crack Cocaine

LSD

Ecstasy

Addiction:

Dopamine

Naloxone

Beta Endorphin

Other Addictive Substances:

Alcohol

Nicotine

Other Substances:

Endorphins

Beta Endorphin

Pheromones

Vitamins:

Vitamins A - E

Chemical Medical Conditions:

Schizophrenia

Dopamine

Alzheimer's

ACh (Acetycholine)

Parkinson's

Dopamine

Depression

PEA (Phenylethylamine)

When Depressed, People Have High Levels of:

Cortisol

ACTH (AdrenoCortico Tropic Hormone)

CRH (Corticotropin Releasing Hormone)

Diabetes

Insulin

Multiple Sclerosis

Interferon

Lifestyle:

Sun Tanning

Melanin

Negative Emotions:

Hate/Anger/Aggression

Noradrenaline (and/or Adrenaline)

Testosterone (increases aggression)

Serotonin (decreases aggression)

Lust

Jealousy

Envy

Fear

Noradrenaline (and/or Adrenaline)

Make photographic representations and create a composite image.

For instances in which the subject materials are chemical compounds, each chemical is prepared by dissolving it with a small quantity of water, or suitable reagent, onto a microscope slide. After the liquid has evaporated, the amalgamated clear crystals are viewed under a microscope using polarized light. The appearance of the crystals is similar to a frosted window pane, and the patterns can be random and unpredictable, but are nonetheless distinguishable from one another, such that the image of one chemical will have certain characteristic details that will not be duplicated in other image components.

The preparation of the chemicals is accomplished using straightforward known crystallographic techniques, and so the details of the specific chemical preparations have been omitted as being well-known to those of ordinary skill in the art of crystallography. However, gauging the relative proportions of chemical to water is generally accomplished by trial and error, to provide sufficient concentration of the desired chemical to provide a dense, content-filled image. While still liquid, a small cover slip is placed on top of the solution. This slows the rate of evaporation of the liquid and allows the crystal lattice time to develop in more uniform and aesthetically pleasing arrangements. Preparation of the chemicals is accomplished at ambient room temperature.

Formation of the crystals can range from minutes to months. Once crystallized, some chemicals may react with atmospheric oxygen, causing clouding of the image. This effectively de-polarizes the light and therefore necessitates the rapid examination and photography of the preparation.

The use of two crossed-polarizing filters allows the display of a property of the crystals known as birefringence. This effect can sometimes be seen in the stress-patterns on laminated automobile windshields when viewed through polarized sunglasses. All the colors that are produced, as shown in the various figures are, therefore, in a sense, artificial, and do not represent an inherent or “absolute” color for the crystal.

The image components will be magnified within a range of 30× to 60×. Higher magnifications will usually be necessary only when the crystals are very small, however, this often yields softer images due to the shallow depth of field of higher power optics, and in preferred embodiments of the invention, higher power magnifications should be avoided whenever possible. In addition, at higher magnifications, the crystal lattices show poorly defined boundary edges, further adding to a softer appearance.

The images are then preferably further enhanced through the use of a three-dimensional (3D) computer graphic program, called LightWave (or any similar tool), the images will then be processed to provide a relief-map appearance to the crystals.

Accordingly, in the embodiment of the “eye” image (FIGS. 35 and 36), first a 3D model of the human eye is created. The image components are then “mapped” around the iris. The iris is then deformed using a 3D “displacement map” which is derived from the images so that each shape and contour of the original acquires an additional depth dimension. This displacement map is created from a monochrome copy of the crystal images by taking the bright and dark parts of the image to form the peaks and valleys of the final relief map.

When combined, the image and the displacement map of that image, produce a photorealistic 3D representation of a previously 2D image.

The selection of the colors for the image components is, in some respects, arbitrary. Indeed, depending upon the desired application of the image, the respective or collective image components may be monochromatic, or even in black and white. Preferably, upon execution of the image components, they will have a minimum resolution of 300 dots per inch (dpi).

For instances in which the subject materials are more complex/more macroscopic in nature (e.g., viruses, bacteria, cellular structures, etc.), standard laboratory photographic techniques may be employed to generate the basic image, which may then be manipulated in terms of color, resolution, or adjustment to produce a simulated three-dimensional effect, as discussed hereinabove.

While an image may be created using a single image component, it is believed that the “impact” of the image will be enhanced through its combination with other related relevant image components. This belief may be based upon the rationale that by advising the view of the identity and significance of each of the image components, the psycho-physiological effects (placebo effect) will be enhanced. An alternative rationale, based upon the “alternative science” theory of resonance, holds that by having an image having a plurality of components, the combination of which is representative of or otherwise related to a specific target state, the “resonance effect” will be greater than if a single image component were used, given that a single material that is used to make a single image component may or may not be unique to that specific target state (i.e., there may not be a one-to-one correspondence relationship between the material and the target state).

Once the image components have been created, then they are assembled together in a configuration which preferably will be aesthetically pleasing (if that is appropriate to the target “state”), or otherwise configured to have maximum impact.

Reference is made earlier to the “eye” composite image. For the state of “love”, it is believed that an image having the overall shape and configuration of a human eye (classically referred to as “the windows to the soul”) is a useful tool for providing a basis for presenting the image components for “love”. From the viewpoint of the “placebo” theory, the aforementioned metaphor adds a further contextual weight to the image components themselves. From the viewpoint of the “resonance” theory, a more literal interpretation of the metaphor is implied. FIG. 35 illustrates an “eye” image composed of oxytocin, serotonin, beta-endorphin, vasopressin, testosterone, estradiol, and noradrenaline. FIG. 36 illustrates an alternative “eye” image. Greater or fewer numbers of chemicals may be selected, as mentioned elsewhere herein, although 6 or 7 of the most salient materials are believed to be an optimum range.

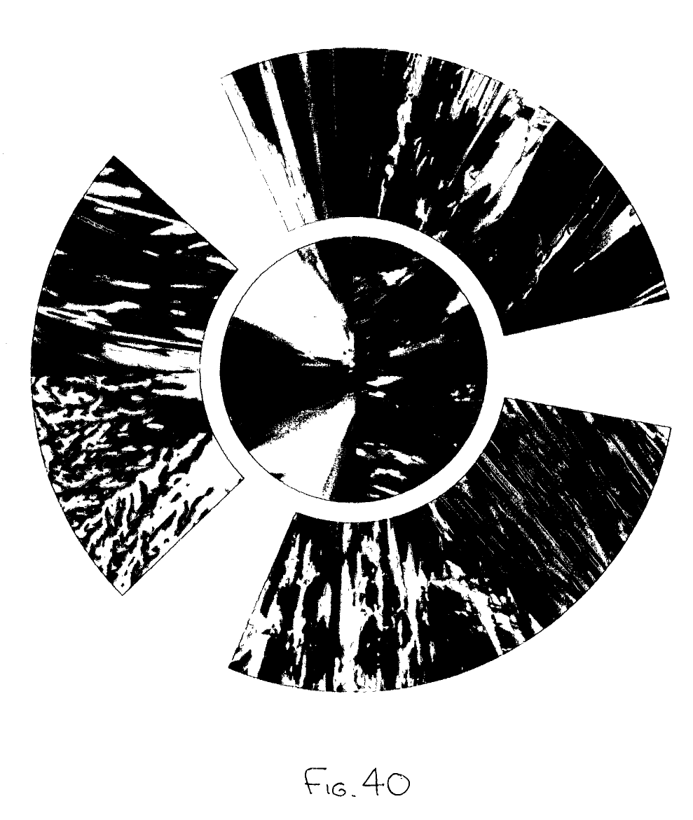

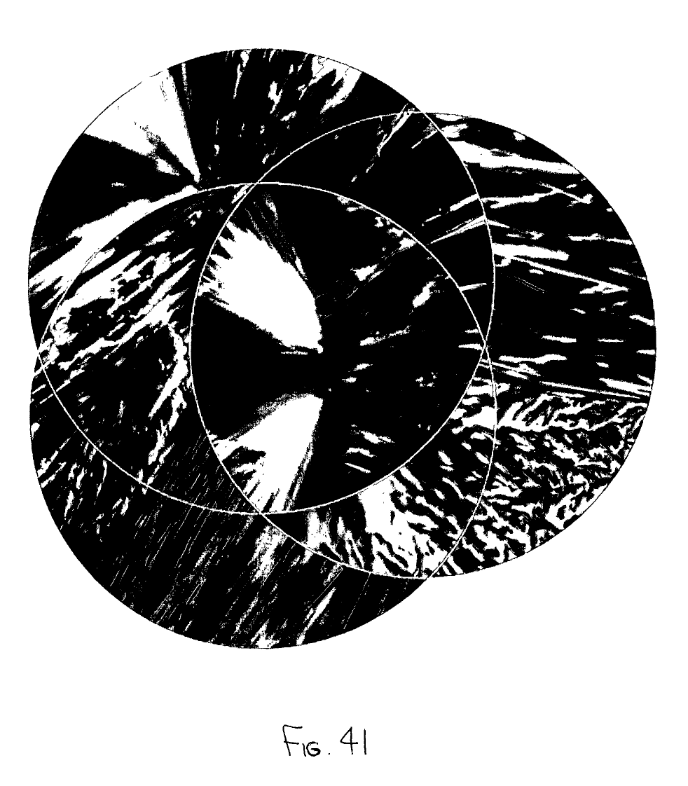

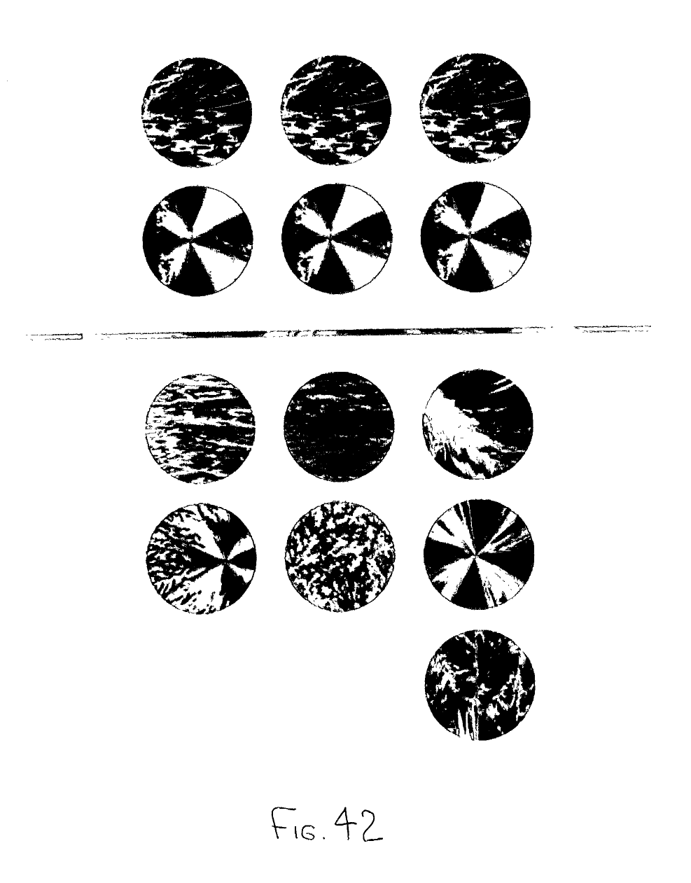

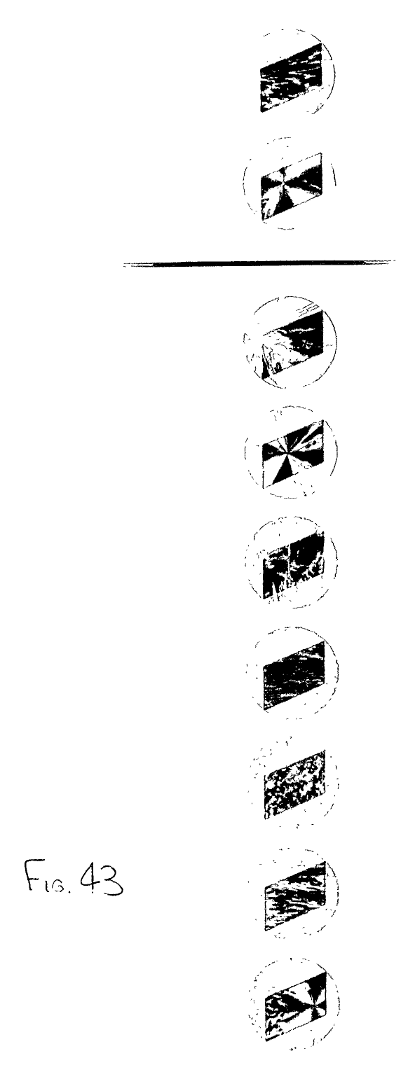

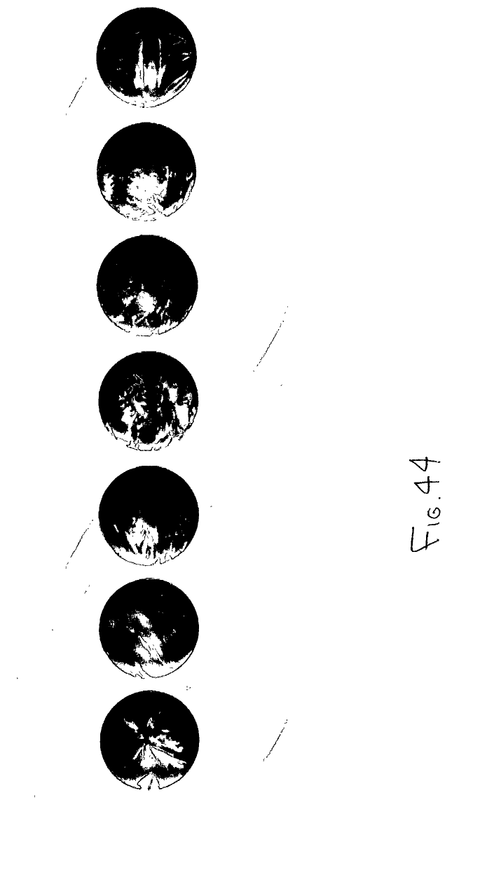

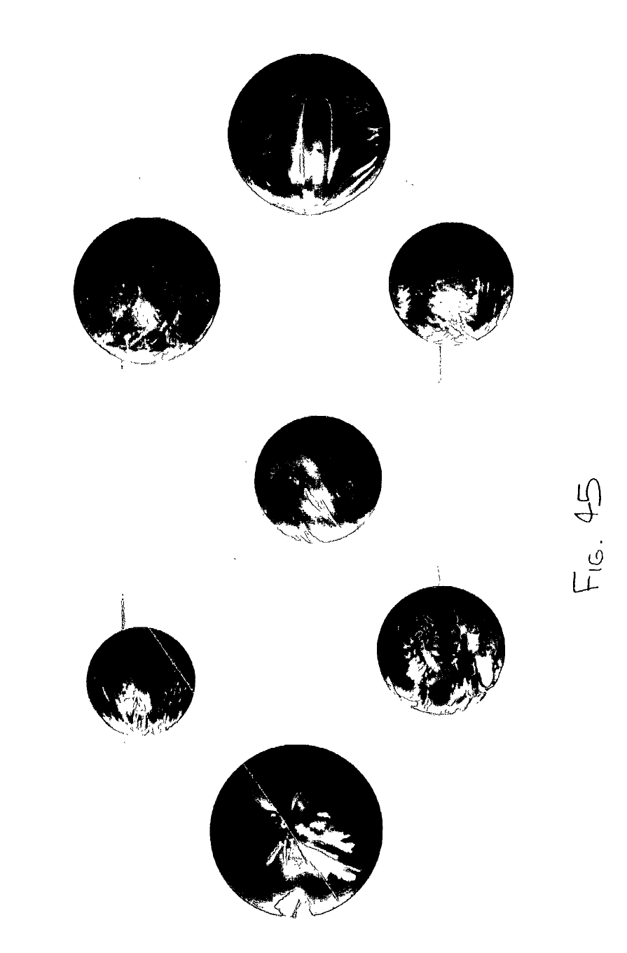

Although the “eye” is a powerful and preferred configuration, other configurations may be used for composite images, which may be more or less representational in nature. FIGS. 37-45 illustrate just some potential image collages. In general, the composite image may be a representation of a human body part (eye, face, silhouette of a head or entire body); a stylized design (such as a conventional “heart” shaped outline or fleur de is); an animal (fish, bird, horse, etc.); a plant (outline of a rose, palm, etc.); a geographical structure (e.g., map representation of a country or geographical feature; silhouette of a geographical feature, such as the Matterhorn, the Rock of Gibraltar, Ayers Rock, etc.); or an abstract geometric figure (circle, square, diamond, rhombus, swirl or spiral, etc.), among other shapes.

Place the composite image on articles. Depending upon the nature of the “target” state, and the objective “audience” for the message (“resonance”) being transmitted, the composite image may be placed on an almost infinite variety of articles, in addition to presentation of the image as a simple “poster” or the like. However, preferred examples of articles upon which the composite images might be placed include garments (e.g., sports clothing); utilitarian articles of all sorts (ranging from vehicles, major appliances, etc.); personal accessories (purses, backpacks, luggage, portfolios, etc.); windows or other transparent/translucent media; contact lenses, eyeglasses and sunglasses; buildings or other outdoor structures; packaging for all the foregoing, as well as for consumer perishable items (packaged foodstuffs, etc.). This list is not and cannot be exhaustive. While in preferred embodiments of the invention, the “message” will be logically related to the “medium” for maximum effect (i.e., “sport” related images on athletic garments and gear; “health” related images on medical supplies, equipment, or as displays in hospitals or clinics, etc.), these images can be placed anywhere it is desired that the “message” be seen, to impact upon a particular viewer's state.

It may be desirable to provide for a collective group of different images, for addressing different mental/emotional/physical states, which are arranged on different articles of a kind (e.g., a suite of “sport” image bearing garments for different occasions, for example garments with “endurance” images for marathon race running, and other garments with “speed” images for sprinting activities, etc.) so that the garments or other articles can be “fine-tuned”. Alternatively, articles may be provided having “all-inclusive” all-purpose images.

As a related scheme, it has been noted that color has a psychological relationship to mental/emotional states. That is, certain colors are typically associated with or representative or reminiscent of, certain emotional states (red for anger, black for depression, gray for melancholy). In another aspect of the present invention, articles (such as garments or ornamentation) may be provided with specifically selected images of a single color or group of colors, for purposes of expressing an existing mental state of a wearer, for seeking to modify or modulate an existing mental state to a desired other mental state of the wearer, or for seeking to modify or modulate an existing mental state to a desired other mental state of those observing and/or interacting with the wearer. An individual may have an entire suite of coordinated garments, from which they may select, in order to address specific mental or even physical states.

In addition to placing the composite image on articles in the manner of a permanent marking or applique, the composite image may also be projected onto an article, such as a building structure or movie screen (as part of a presentation or advertisement); may be presented to the intended subject via print, videotaped and replayed images; television broadcast or cable image; or via email or internet website.

It is noted that because there are many influence materials that are representative of a wide variety of emotions, physical or mental states, that the various images that may be made from them can, in fact, be used to form a rudimentary “language” whereby selecting a sequence of images or combinations of images, based upon their representative corresponding “meanings” can be used to formulate and “communicate” more complex messages, analogous to semaphore or the like.

The foregoing description and drawings merely explain and illustrate the invention, and the invention is not limited except insofar as the appended claims are so limited, as those skilled in the art who have the disclosure before them will be able to make modifications and variations therein without departing from the scope of the invention.

We claim:

1. A method for influencing an emotional or physical state of an individual, using a visual image, comprising the steps of:
identifying a particular target state, being at least one of mental, emotional, physical, to be influenced;
identifying the desired manner in which the particular target state is to be influenced;
identifying at least one influence material which is one of causative of, resultant of, the target state;
making at least one visual representation of the at least one influence material;
creating at least one image component from each at least one visual representation of the at least one influence material;
creating a composite image from the at least one image component;
placing the composite image on an article;
bringing the individual to be influenced into contact with the article.

2. The method according to claim 1, wherein the step of identifying the desired manner in which the particular target state is to be influenced further comprises the step of:
determining whether the influence sought is to be to maintain the target state, promote the target state, or prevent the target state.

3. The method according to claim 1, wherein the influence material is one of:
a chemical which when brought into interactive contact with an individual directly influences the target state; a chemical which is found in an individual when that individual is experiencing the target state and which is characteristic of that target state; a cellular structure; an organism; an object.

4. The method according to claim 1, wherein the step of making at least one visual representation of the at least one influence material further comprises the step of:
making a photographic representation of the at least one influence material.

5. The method according to claim 1, wherein the step of creating at least one image component from each at least one visual representation of the at least one influence material further comprises the step of:
selectively altering the color values of the photographic representation.

6. The method according to claim 1, wherein the step of creating at least one image component from each at least one visual representation of the at least one influence material comprises the step of:
imposing a selected outline shape for the at least one image component.

7. The method according to claim 1, wherein the step of creating at least one image component from each at least one visual representation of the at least one influence material further comprises the step of:
creating at least two image components from at least two visual representations of at least one influence material.

8. The method according to claim 7, wherein the step of creating a composite image further comprises the step of:
selectively positioning the at least two image components relative to one another.

9. The method according to claim 1, wherein the step of creating at least one image component from each at least one visual representation of the at least one influence material further comprises the step of:
creating at least two image components from at least two visual representations of at least two influence materials.

10. The method according to claim 1, wherein the step of creating a composite image further comprises the step of:
selectively positioning the at least two image components relative to one another; and
imposing a selected shape to the contours of the selectively positioned at least two image components, the selected shape having an association with the target state to be influenced.

11. The method according to claim 1, wherein the article upon which the composite image is to be placed comprises one of:
a sheet of opaque display media; a sheet of transparent media; a sheet of translucent media; printing media; a garment; a structure; a vehicle; packaging for a commercial product; a visual prosthetic aid device.

12. The method according to claim 1, further comprising the step of:
associating verbal information content with the visual image, identifying the nature of the at least one image component.

13. A method of preparation of a visual image for influencing an emotional or physical state of an individual, using the visual image, comprising the steps of:
identifying a particular target state, being at least one of mental, emotional, physical, to be influenced;
identifying the desired manner in which the particular target state is to be influenced;
identifying at least one influence material which is one of causative of, resultant of, the target state;
making at least one visual representation of the at least one influence material;
creating at least one image component from each at least one visual representation of the at least one influence material;
creating a composite image from the at least one image component.

14. The method according to claim 13, wherein the step of identifying the desired manner in which the particular target state is to be influenced further comprises the step of:
determining whether the influence sought is to be to maintain the target state, promote the target state, or prevent the target state.

15. The method according to claim 13, wherein the influence material is one of:
a chemical which when brought into interactive contact with an individual directly influences the target state; a chemical which is found in an individual when that individual is experiencing the target state and which is characteristic of that target state; a cellular structure; an organism; an object.

16. The method according to claim 13, wherein the step of making at least one visual representation of the at least one influence material further comprises the step of:
making a photographic representation of the at least one influence material.

17. The method according to claim 13, wherein the step of creating at least one image component from each at least one visual representation of the at least one influence material further comprises the step of:
selectively altering the color values of the photographic representation.

18. The method according to claim 13, wherein the step of creating at least one image component from each at least one visual representation of the at least one influence material comprises the step of:
imposing a selected outline shape for the at least one image component.

19. The method according to claim 13, wherein the step of creating at least one image component from each at least one visual representation of the at least one influence material further comprises the step of:
creating at least two image components from at least two visual representations of at least one influence material.

20. The method according to claim 19, wherein the step of creating a composite image further comprises the step of:
selectively positioning the at least two image components relative to one another.

21. The method according to claim 13, wherein the step of creating at least one image component from each at least one visual representation of the at least one influence material further comprises the step of:
creating at least two image components from at least two visual representations of at least two influence materials.

22. The method according to claim 13, wherein the step of creating a composite image further comprises the step of:
selectively positioning the at least two image components relative to one another; and
imposing a selected shape to the contours of the selectively positioned at least two image components, the selected shape having an association with the target state to be influenced.

23. The method according to claim 13, wherein the article upon which the composite image is to be placed comprises one of:
a sheet of opaque display media; a sheet of transparent media; a sheet of translucent media; printing media; a garment; a structure; a vehicle; packaging for a commercial product; a visual prosthetic aid device.

24. An article bearing a visual image for influencing an emotional or physical state of an individual, using the visual image, comprising:
a physical object;
a composite image disposed thereon,
the composite image including at least one image component,
the at least one image component including at least one visual representation of an influence material,
the at least one influence material being one of causative of, resultant of, a target state of an individual which is to be influenced,
the target state of the individual which is to be influenced, being one of mental, emotional, physical.

25. The article according to claim 24, wherein the physical object is one of:
a sheet of opaque display media; a sheet of transparent media; a sheet of translucent media; printing media; a garment; a structure; a vehicle; packaging for a commercial product; a visual prosthetic aid device.

26. The article according to claim 23, wherein the at least one influence material is one of:
a chemical which when brought into interactive contact with an individual directly influences the target state; a chemical which is found in an individual when that individual is experiencing the target state and which is characteristic of that target state; a cellular structure; an organism; an object.

27. The method according to claim 13, further comprising one of the following steps:
projecting the composite image onto an article;
publishing the composite image in a printed medium;
recording the composite image onto videotape;
broadcasting the composite image via one of television, cable, satellite communication;
communicating the composite image via email;
locating the composite image in an internet website.
